(19) United States
(12) Patent Application Publication
(10) Pub. No.: US 2026/0345192 A1
(43) Pub. Date: Nov. 5, 2026

(54) RAPID SKIN TREATMENT USING MICROCORING

(71) Applicant: Cytrellis Biosystems, Inc., Woburn, MA (US)

(72) Inventors: Douglas Levinson, Sherborn, MA (US); Karen Cronholm, Arlington, MA (US); Alec Ginggen, Medford, MA (US)

(21) Appl. No.: 19/392,713

(22) Filed: Nov. 18, 2025

Related U.S. Application Data
(63) Continuation of application No. 16/857,801, filed on Apr. 24, 2020, now abandoned.
Application No. 16/857,801 is a continuation of application No. 15/711,943, filed on Sep. 21, 2017, now abandoned.
(60) Provisional application No. 62/397,865, filed on Sep. 21, 2016.

(51) Int. Cl. A61B 17/3205; A61B 5/00; A61B 5/24; A61B 10/02; A61B 17/00; A61M 37/00

(52) U.S. Cl. CPC A61B 17/32053; A61B 5/24; A61B 10/0233; A61B 10/0266; A61B 10/0283; A61B 5/441; A61B 2010/0225; A61B 2017/00053; A61B 2017/00747; A61B 2017/00761; A61B 2017/00792; A61M 37/0015; A61M 2037/0023; A61M 2037/0061

(57) ABSTRACT

Described herein are technologies, methods, and/or devices for treating skin (e.g., eliminating tissue volume, tightening skin, lifting skin, and/or reducing skin laxity) by selectively excising a plurality of microcores without thermal energy being imparted to surrounding (e.g., non-excised) tissue. In certain embodiments, excising is completed within a certain time or is performed at a certain rate. In certain embodiments, treatment is performed in specific areas not treatable with thermal methods, e.g., in the vicinity of nerves and/or other heat sensitive areas. In certain embodiments, a cosmetic effect is visible immediately or within a very short time after completion of treatment.

### RELATED APPLICATIONS

This application is a continuation of U.S. patent application Ser. No. 16/857,801 (Docket No.: CYT-009-US-CON1), titled “Rapid Skin Treatment Using Microcoring”, filed Apr. 24, 2020, United States Publication No. 2020/0246039, published Aug. 6, 2020, which is a continuation of U.S. patent application Ser. No. 15/711,943 (Docket No.: CYT-009-US), titled “Rapid Skin Treatment Using Microcoring”, filed Sep. 21, 2017, United States Publication No. 2018/0078278, published Mar. 22, 2018, which claims the benefit of U.S. Provisional Patent Application Ser. No. 62/397,865 (Docket No.: CYT-009-PR), titled “Rapid Skin Treatment Using Microcoring”, filed Sep. 21, 2016, the content of which is incorporated herein by reference in its entirety for all purposes.

### BACKGROUND

Many human health issues arise from the damage, deterioration, or loss of tissue due to disease, advanced age, and/or injury. These health issues can manifest themselves in a variety of alterations of tissue structure and/or function, including scarring, sclerosis, tightness, and laxity. In aesthetic medicine, elimination of excess tissue and/or skin laxity is an important concern that affects more than 25% of the U.S. population. In a recent survey (September 2015) of 1052 women in the US (ages 35-75), 78% of women surveyed felt that they had sagging skin, and 83% of these women were self-conscious about it. In addition, 86% of women surveyed felt that they had wrinkles.

Conventional surgical therapies (e.g., a face lift, brow lift, or breast lift) can be effective in treating a wide variety of skin/tissue conditions, but are often invasive, inconvenient, and expensive. Invasive techniques carry elevated risks of side effects and often require prolonged healing times. These techniques often require trained physicians and nurses, and need to be carried out in a surgical environment. In addition, most conventional therapies require significant physician time, which is a main factor for the costs of these treatments, as well as significant preparation and/or healing times. These disadvantages of current techniques are reflected in the results of a recent survey (September 2015), wherein 74% of women surveyed would not consider having surgery to address their concerns of skin laxity. Additionally, scarring limits the applicability of surgery to certain treatment sites. Although minimally invasive methods are available, such methods are generally less effective than surgical methods. Surgical therapies remain the gold standard for lifting and/or tightening skin, as compared to energy-based techniques and injection-based techniques.

Injection-based techniques are available, but neurotoxins, such as botulinum toxin, have minimal or no direct effect on skin tightness or laxity, and dermal fillers, such as hyaluronic acid, do not directly tighten or reduce laxity of the skin.

Methods using energy sources (e.g., laser, non-coherent light, radiofrequency, or ultrasound) can be effective at improving the architecture and the texture of the skin, but are much less effective at tightening the skin or reducing skin laxity. In addition, energy-based methods carry the risk of side effect (e.g., burns, skin bleaching, nerve damage etc.), which greatly reduces their applicability.

Accordingly, there is a need for improved technologies, methods, and/or devices that combine the effectiveness of surgical interventions with convenience and speed of minimally-invasive techniques while significantly reducing side effects, improving preparation, aftercare, and healing time, as well as increasing the range of applicable tissue types and patient populations.

### SUMMARY

Described herein are technologies, methods, and/or devices for treating skin (e.g., eliminating tissue volume, tightening skin, lifting skin, and/or reducing skin laxity) by selectively excising a plurality of microcores without thermal energy being imparted to surrounding (e.g., non-excised) tissue. The technologies, methods, and/or devices described herein satisfy an unmet need for rapid and safe treatment of skin, including, e.g., faster pre-treatment preparation and post-treatment healing times compared to current surgical and thermal treatment methods.

In one aspect, the invention is directed to a method comprising steps of excising a plurality of microcores from a site on a surface of a human subject, wherein each of the microcores is characterized by a diameter of between 0.1 mm and 1.0 mm, and/or a volume of between 0.001 mm³ and 6.3 mm³, wherein the excising is completed within a time period between 1 minute and 2 hours.

In one aspect, the invention is directed to a method comprising steps of: excising a plurality of microcores from a site on a surface of a human subject, wherein each of the microcores is characterized by a diameter of between 0.1 mm and 1.0 mm, and a volume of between 0.001 mm³ and 6.3 mm³, wherein the excising is performed at a rate of between 100 to 30,000 cores/minute, or is performed in a single application of a needle array.

In some embodiments, the microcores are sequestered.

In some embodiments, the sequestered microcores are discarded and/or used for diagnostics.

In some embodiments, wherein the plurality of microcores comprises at least 1,500, at least 10,000, or at least 100,000 microcores.

In some embodiments, wherein the site has dimensions of between 1 cm² and 300 cm² between 1.2 cm² and 280 cm², between 1.4 cm² and 260 cm², between 1.6 cm² and 240 cm², between 1.8 cm² and 220 cm², between 2 cm² and 200 cm², between 2.2 cm² and 180 cm², between 2.4 cm² and 160 cm², between 2.6 cm² and 140 cm², between 2.8 cm² and 120 cm² between 3 cm² and 100 cm², between 3.2 cm² and 80 cm², between 3.4 cm² and 60 cm², between 3.6 cm² and 40 cm², between 3.8 cm² and 20 cm², between 4 cm² and 10 cm², between 10 cm² and 20 cm², between 20 cm² and 30 cm², between 30 cm² and 40 cm², between 40 cm² and 50 cm², between 50 cm² and 60 cm², between 60 cm² and 70 cm², between 70 cm² and 80 cm², between 80 cm² and 100 cm², between 100 cm² and 120 cm², between 120 cm² and 140 cm², between 140 cm² and 160 cm², between 160 cm² and 180 cm², or between 180 cm² and 200 cm².

In some embodiments, wherein the surface is selected from the group consisting of the face, eyelid, cheeks, chin, forehead, lips, or nose, neck, chest, arms, hands, legs, abdomen, buttock, and thigh.

In some embodiments, the diameter is between about 0.14 mm and about 0.84 mm, 0.16 mm and about 0.82 mm, 0.18 mm and about 0.8 mm, 0.2 mm and about 0.78 mm, 0.22 mm and about 0.76 mm, 0.24 mm and about 0.74 mm, 0.26 mm and about 0.72 mm, 0.28 mm and about 0.7 mm, 0.3 mm and about 0.68 mm, 0.32 mm and about 0.66 mm, 0.34 mm and about 0.64 mm, 0.36 mm and about 0.62 mm, 0.38 mm and about 0.6 mm, 0.4 mm and about 0.58 mm, 0.42 mm and about 0.56 mm, 0.44 mm and about 0.54 mm, 0.46 mm and about 0.52 mm, or 0.48 mm and about 0.5 mm.

In some embodiments, the volume is between about 0.005 mm³ and 5.0 mm³, 0.01 mm³ and 4.0 mm³, 0.015 mm³ and 3.0 mm³, 0.02 mm³ and 2.0 mm³, 0.022 mm³ and 1.8 mm³, 0.024 mm³ and 1.6 mm³, 0.026 mm³ and 1.4 mm³, 0.028 mm³ and 1.2 mm³, 0.03 mm³ and 1.0 mm³, 0.032 mm³ and 0.8 mm³, 0.034 mm³ and 0.6 mm³, 0.036 mm³ and 0.4 mm³, 0.038 mm³ and 0.2 mm³, 0.04 mm³ and 0.1 mm³, or 0.06 mm³ and 0.08 mm³.

In some embodiments, each of the microcores is characterized by a length of between 0.3 mm and 6.2 mm (e.g., between about 0.3 mm and 0.6 mm, 0.3 mm and 0.9 mm, 0.3 mm and 1.5 mm, 0.3 mm and 2.0 mm, 0.3 mm and 2.5 mm, 0.3 mm and 3.0 mm, 0.3 mm and 3.5 mm, 0.3 mm and 4.0 mm, 0.3 mm and 4.5 mm, 0.3 mm and 5.0 mm, 0.3 mm and 5.5 mm, 0.3 mm and 6.0 mm, 0.3 mm and 6.2 mm, 0.6 mm and 0.9 mm, 0.6 mm and 1.5 mm, 0.6 mm and 2.0 mm, 0.6 mm and 2.5 mm, 0.6 mm and 3.0 mm, 0.6 mm and 3.5 mm, 0.6 mm and 4.0 mm, 0.6 mm and 4.5 mm, 0.6 mm and 5.0 mm, 0.6 mm and 5.5 mm, 0.6 mm and 6.0 mm, 0.6 mm and 6.2 mm, 0.9 mm and 1.5 mm, 0.9 mm and 2.0 mm, 0.9 mm and 2.5 mm, 0.9 mm and 3.0 mm, 0.9 mm and 3.5 mm, 0.9 mm and 4.0 mm, 0.9 mm and 4.5 mm, 0.9 mm and 5.0 mm, 0.9 mm and 5.5 mm, 0.9 mm and 6.0 mm, 0.9 mm and 6.2 mm, 1.5 mm and 2.0 mm, 1.5 mm and 2.5, mm, 1.5 mm and 3.0 mm, 1.5 mm and 3.5 mm, 1.5 mm and 4.0 mm, 1.5 mm and 4.5 mm, 1.5 mm and 5.0 mm, 1.5 mm and 5.5 mm, 1.5 mm and 6.0 mm, 1.5 mm and 6.2 mm, 2.0 mm and 2.5 mm, 2.0 mm and 3.0 mm, 2.0 mm and 3.5 mm, 2.0 mm and 4.0 mm, 2.0 mm and 4.5 mm, 2.0 mm and 5.0 mm, 2.0 mm and 5.5 mm, 2.0 and 6.0 mm, 2.0 mm and 6.2 mm, 2.5 mm and 3.0 mm, 2.5 mm and 3.5 mm, 2.5 mm and 4.0 mm, 2.5 mm and 4.5 mm, 2.5 mm and 5.0 mm, 2.5 mm and 5.5 mm, 2.5 mm and 6.0 mm, 2.5 mm and 6.2 mm, 3.0 mm and 3.5 mm, 3.0 mm and 4.0 mm, 3.0 mm and 4.5 mm, 3.0 mm and 5.0 mm, 3.0 mm and 5.5 mm, 3.0 and 6.0 mm, 3.0 mm and 6.2 mm, 3.5 mm and 4.0 mm, 3.5 mm and 4.5 mm, 3.5 mm and 5.0 mm, 3.5 mm and 5.5 mm, 3.5 and 6.0 mm, 3.5 mm and 6.2 mm, 4.0 mm and 4.5 mm, 4.0 mm and 5.0 mm, 4.0 mm and 5.5 mm, 4.0 and 6.0 mm, 4.0 mm and 6.2 5 mm, 4.5 mm and 5.0 mm, 4.5 mm and 5.5 mm, 4.5 and 6.0 mm, 4.5 mm and 6.2 mm, 5.0 mm and 5.5 mm, 5.0 mm and 6.0 mm, 5.0 mm and 6.2 mm, 5.5 mm and 6.0 mm, 5.5 mm and 6.2 mm, or 6.0 mm and 6.2 mm).

In some embodiments, each of the microcores is characterized by a length of between 6.2 mm and 9.2 mm, 6.2 mm and 9 mm, 6.2 mm and 8.8 mm, 6.2 mm and 8.6 mm, 6.2 mm and 8.4 mm, 6.2 mm and 8.2 mm, 6.2 mm and 8 mm, 6.2 mm and 7.8 mm, 6.2 mm and 7.6 mm, 6.2 mm and 7.4 mm, 6.2 mm and 7.2 mm, 6.2 mm and 7 mm, 6.2 mm and 6.8 mm, 6.2 mm and 6.6 mm, or 6.2 mm and 6.4 mm.

In some embodiments, a length of the micorcore is sufficient to obtain a full thickness core.

In some embodiments, a length of the micorcore is sufficient to extend into the subcutaneous fat layer.

In some embodiments, the time period is between 2 minutes and 1.5 hours, between 3 minutes and 1.2 hours, between 4 minutes and 1 hour, between 5 minutes and 50 minutes, between 6 minutes and 45 minutes, between 7 minutes and 40 minutes, between 8 minutes and 35 minutes, between 9 minutes and 30 minutes, or between 10 minutes and 25 minutes.

In some embodiments, the rates are between about 120 and about 25,000 cores/min, about 140 and about 20,000 cores/minute, about 160 and about 15,000 cores/minute, about 180 and about 10,000 cores/minute, about 200 and about 5,000 cores/minute, about 220 and about 4,000 cores/minute, about 220 and about 3,000 cores/minute about, 240 and about 2,000 cores/minute, about 260 and about 1,000 cores/minute, about 280 and about 900 cores/minute, about 300 and about 800 cores/minute, about 320 and about 700 cores/minute, about 340 and about 600 cores/minute, about 360 and about 500 cores/minute, or about 380 and about 400 cores/minute.

In some embodiments, the surface is the face and the time period is between 15 minutes and 30 minutes.

In some embodiments, the needle array comprises between 10 and 100,000 needles, between 20 and 50,000 needles, between 30 and 25,000 needles, between 40 and 15,000 needles, between 50 and 10,000 needles, between 60 and 8,000 needles, between 70 and 6,000 needles, between 80, and 4,000 needles, between 90 and 2,000 needles, between 100 and 1,000 needles, between 120 and 800 needles, between 140 and 600 needles, or between 160 and 400 needles.

In some embodiments, the area or volumetric fraction of tissue excised from the site is between 0.1% and 65% of the area of the site.

In some embodiments, the area or volumetric fraction of tissue excised from the site is 10% of the area of the site.

In some embodiments, the microcores are excised without excising the epidermal layer.

In some embodiments, the site is pre-treated prior to receiving treatment using microcoring, wherein the pre-treatment comprises elevating and/or stretching the skin.

In some embodiments, the method comprises determining the presence of a nerve beneath the surface of a site prior to removing/excising a microcore.

In some embodiments, the presence of a nerve is determined via dynamic sensing.

In some embodiments, the presence of a nerve is determined via detection using a feedback sensor, wherein the sensor detects transition from one dermal layer to another.

In some embodiments, the presence of a nerve is determined via detection using nerve excitation.

In some embodiments, the presence of a nerve is determined via mapping.

In some embodiments, the site is a heat-sensitive site or a light/UV-sensitive site.

In some embodiments, the site is a heat-sensitive site.

In some embodiments, the site is a light/UV-sensitive site.

In some embodiments, the site is located on the face.

In some embodiments, the site is located on the neck.

In some embodiments, the site is located on the face in close proximity to an eye.

In some embodiments, the site is located on the face in close proximity to the facial nerve or a facial nerve branch.

In some embodiments, the facial nerve branch is the temporal branch, the zygomatic branch, the buccal branch, the marginal mandibular branch, or the cervical branch.

In some embodiments, the site is located over an area that comprises a mechanical implant, a dermal filler, or a breast implant.

In some embodiments, the site is located over or near the thyroid gland, thyroid cartilage, trachea, a major blood vessel, or breast tissue.

In some embodiments, the site is not located over an area that comprises a mechanical implant, a dermal filler, or a breast implant.

In some embodiments, the site is not located over or near the thyroid gland, thyroid cartilage, trachea, a major blood vessel, or breast tissue.

In some embodiments, the method comprises separating the dermal layer from the superficial muscular aponeurotic system (SMAS) layer.

In some embodiments, the method does not comprise separating the dermal layer from the SMAS layer.

In some embodiments, the method comprises removing the SMAS layer.

In some embodiments, the subject has been treated with ultrasound therapy, laser therapy, radiofrequency, botox, dermafillers, or cosmetic surgery prior to receiving treatment using microcoring.

In some embodiments, the subject has not been treated with ultrasound therapy, laser therapy, radiofrequency, botox, dermafillers, or cosmetic surgery prior to receiving treatment using microcoring.

In some embodiments, the subject is between 40-70 years of age; has Fitzpatrick Skin Type 1, 2, or 3; has re-auricular wrinkle severity graded as ≥2 and/or one or more of the following: Nasolabial fold severity at rest ≥2 and ≤4; Marionette line prominence at rest ≥2 and ≤4; Oral commissure drooping at rest ≥2 and ≤4; or Jawline sagging at rest ≥2 and ≤4.

In some embodiments, the subject does not have any of: lesions suspicious for any malignancy or the presence of actinic keratosis, melasma, vitiligo, cutaneous papules/nodules or active inflammatory lesions in the areas to be treated; history of keloid formation or hypertrophic scarring; history of trauma or surgery to the treatment areas; scar present in the areas to be treated; silicone or synthetic material injections in the areas to be treated; injection of FDA-approved dermal fillers in the past two years; injection of fat in the past year; history of treatment with dermabrasion, laser, or radiofrequency; history of treatment with botulinum toxin injections in the areas to be treated within the prior 6 months; active, chronic, or recurrent infection; history of compromised immune system or currently being treated with immunosuppressive agents; history of sensitivity to analgesic agents, Aquaphor®, topical or local anesthetics (e.g., lidocaine, benzocaine, procaine) or chlorhexidine, povidone-iodine or epinephrine; excessive sun exposure and use of tanning beds or tanning creams within 30 days prior to treatment; treatment with aspirin or other blood thinning agents within 14 days prior to treatment; History or presence of any clinically significant bleeding disorder; and history of drug and/or alcohol abuse; and wherein the subject is not an active smoker (0.5 pack/day) or has quit within 3 months prior to treatment.

In some embodiments, the subject has Fitzpatrick Skin Type 4, 5, or 6.

In some embodiments, the subject, on Day 3 post treatment, experiences ecchymosis, tenderness, pruritis, erythema/inflammation, crusting, hyper pigmentation, hypo pigmentation, swelling/fluid accumulation, and/or bleeding at an average severity level of below 1.5 (on 0-4 severity scale), and wherein the subject exhibits no appearance of scarring.

In some embodiments, the subject, on Day 5 post treatment, experiences ecchymosis, tenderness, pruritis, erythema/inflammation, crusting, hyper pigmentation, hypo pigmentation, swelling/fluid accumulation, and/or bleeding at an average severity level of below 1.5 (on 0-4 severity scale), and wherein the subject exhibits no appearance of scarring.

In some embodiments, the subject, on Day 7 post treatment, experiences a global aesthetic improvement scale (GAIS) score of at least 3 (Improved).

In some embodiments, the subject, on Day 7 post treatment, has re-auricular wrinkle severity improved by at least 1 level; Nasolabial fold severity at rest improved by at least 1 level; Marionette line prominence at rest improved by at least 1 level; Oral commissure drooping at rest improved by at least 1 level; or Jawline sagging at rest improved by at least 1 level.

In some embodiments, the subject, 6 months post treatment, has re-auricular wrinkle severity improved by at least 1 level; Nasolabial fold severity at rest improved by at least 1 level; Marionette line prominence at rest improved by at least 1 level; Oral commissure drooping at rest improved by at least 1 level; or Jawline sagging at rest improved by at least 1 level.

In some embodiments, a cosmetic effect is first detectable during treatment, or immediately after completion of treatment, or 1 min, 5 min, 10 min, 20 min, 30 min, 1 hour, 2 hours, 3 hours, 6 hours, 12 hours, 24 hours, 2 days, 3 days, 4 days, 5 days, 6 days, 7 days, 2 weeks, 3 weeks, 1 month, 2 months, 3 months, 4 months, 5 months, or 6 months after completion of treatment.

In some embodiments, the cosmetic effect is cosmetic skin tightening.

In some embodiments, the cosmetic effect is detectable across the full site.

In some embodiments, cosmetic skin tightening is detectable within a time period no longer than 7 days after completion of treatment.

In some embodiments, the excising is carried out using an apparatus, wherein the apparatus comprises: at least one hollow needle comprising at least a first prong provided at a distal end of the hollow needle, wherein an angle between a lateral side of the first prong and a longitudinal axis of the hollow needle is at least about 20 degrees, and wherein the hollow needle is configured to remove a portion of the skin tissue when the hollow needle is inserted into and withdrawn from the skin tissue.

In some embodiments, the excising is carried out using an apparatus, wherein the apparatus comprises: a needle assembly comprising a hollow needle, a z-actuator, and a tissue removal tool, wherein the hollow needle comprises at least a first prong provided at a distal end of the hollow needle and wherein an angle (α) between a lateral side of the first prong and a longitudinal axis of the hollow needle is at least about 20 degrees.

In one aspect, the invention is directed to a method comprising steps of: excising, using a microcoring implement, a plurality of microcores from a site on a surface of a human subject, comprising microcoring a tissue only below an epidermis layer while leaving the epidermis, and/or other tissue layer above the layer to be excised, un-cored.

In some embodiments, the microcoring implement has a first configuration that allows the microcoring implement to travel through a tissue layer without microcoring said tissue layer, and has a second configuration that allows for the formation of a microcore.

In some embodiments, the microcoring implement has a first configuration resembling a solid needle, and has a second configuration resembling a hollow needle.

### BRIEF DESCRIPTION OF THE DRAWINGS

FIG. 1 shows reference images for assessing pre-auricular wrinkle severity using the Lemperle Assessment Scale. Grading Scale: 0=No wrinkles; 1=Just perceptible wrinkles; 2=Shallow wrinkles; 3=Moderately deep wrinkles; 4=Deep wrinkles, well-defined edges; 5=Very deep wrinkles, redundant fold.

FIG. 2 shows the reference images for assessing nasolabial fold severity at rest using a scale of 1-5.

FIG. 3 shows the reference images for assessing marionette line prominence at rest using a scale of 1-5.

FIG. 4 shows the reference images for assessing oral commissure drooping at rest using a scale of 1-5.

FIG. 5 shows the reference images for assessing jawline sagging at rest using a scale of 1-5.

FIG. 6 shows the treatment area for Part B of the Exemplary study.

### DEFINITIONS

“Animal:” As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

“About:” As used herein, the term “about,” as applied to one or more values of interest, refers to a value that is similar to a stated reference value. In certain embodiments, the term or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Combination therapy:” As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agents or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

“Comprising:” A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

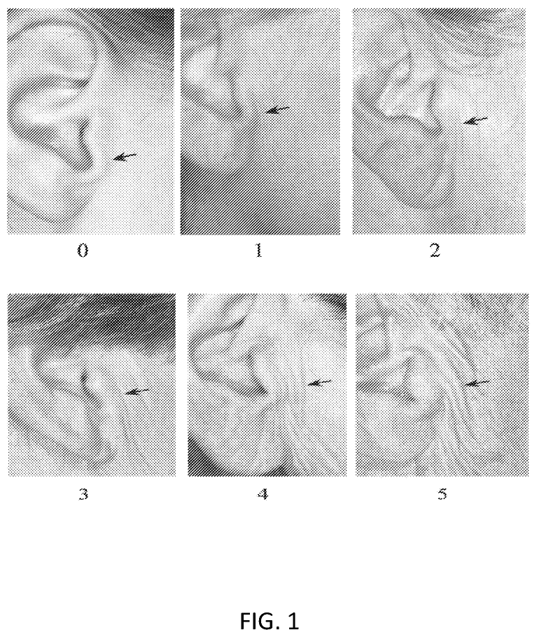

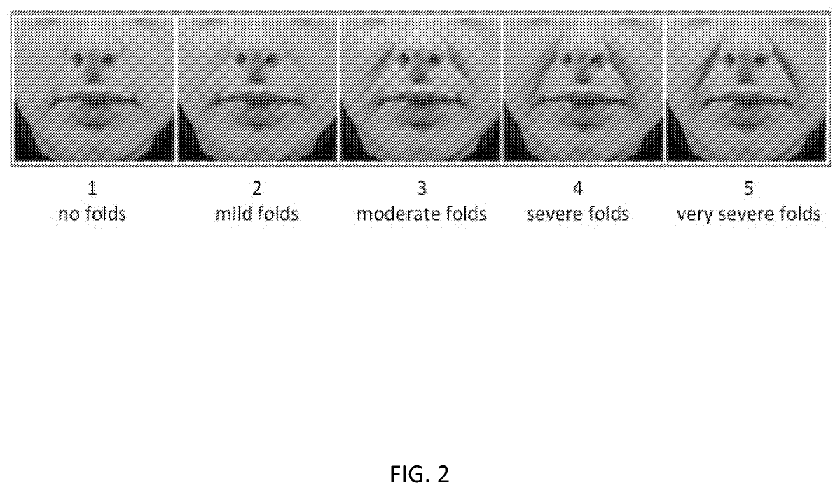

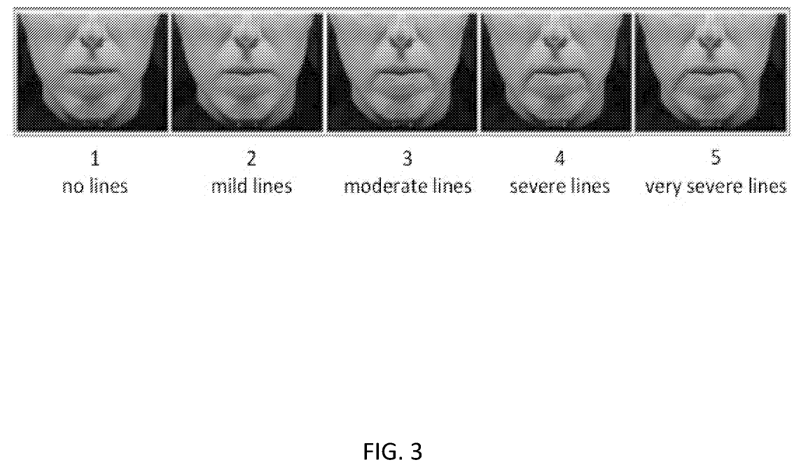

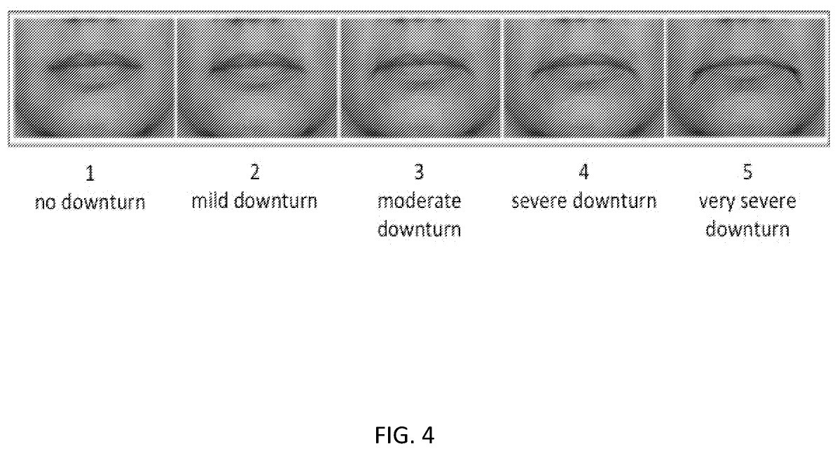

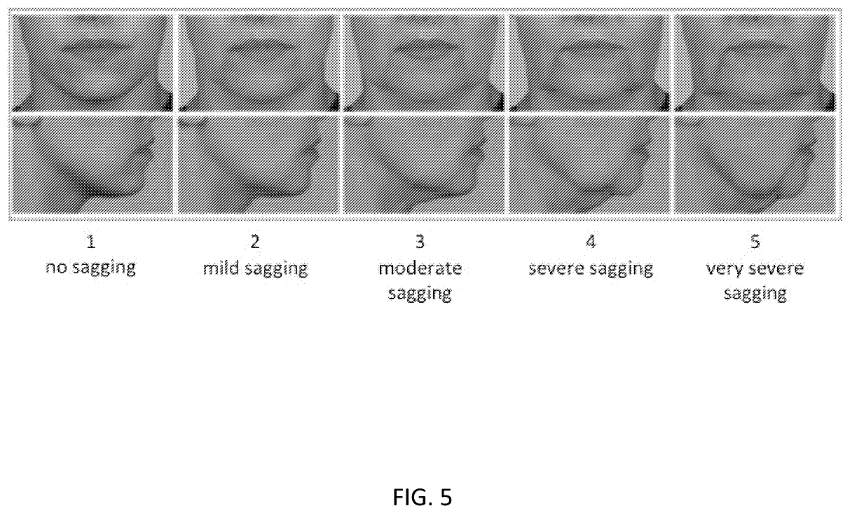

“Cosmetic effect:” As used herein, “cosmetic effect” means a change in a skin appearance, e.g., elimination of tissue volume, tightening of skin, lifting of skin, and/or reduction skin laxity, that is visible, detectable, and/or quantifiable, e.g., a ≥1 point reduction of the Lemperle Scale, e.g., for pre-auricular wrinkles (see FIG. 1), as judged by a Live independent, blinded reviewer; a ≥1 point reduction of the Lemperle Assessment Scale as judged by a 3 member, blinded independent review committee comparing photographs before and after treatment; change in lower face scales as judged by independent, blinded reviewer before and after treatment; Evaluation of photographs of lower face scales by a 3 member independent, blinded review committee before and after treatment; or Evaluation, e.g., by Live independent review or by a 3 member committee, conducted before and after treatment using the following scales: ≥1 point reduction of the nasolabial fold scale score at rest as described in FIG. 2; ≥1 point reduction of the marionette line scale score at rest as described in FIG. 3; ≥1 point reduction of the oral commissure scale score at rest as described in FIG. 4; ≥1 point reduction of the jawline scale score at rest described in FIG. 5.

“Excising:” As used herein, “excising” means a tissue means forming a tissue portion (the “microcore”), e.g., by inserting a hollow needle into the site so that the tissue portion is formed inside the hollow needle and severed from surrounding tissue so that a microcore that is separate from other tissue is generated.

“Full thickness core:” As used herein, “full thickness core” means a microcore whose depth extends through the entire dermal layer beyond the junction of the dermal layer and the subcutaneous fat layer, and into the subcutaneous fat layer.

“Heat sensitive:” As used herein, “heat sensitive” or, e.g., a “heat-sensitive site” means, e.g., a site where exposure to radiation and/or elevated temperature is associated with a relatively high risk of unacceptable cosmetic and/or physiologic outcomes.

“Improve,” “increase” or “reduce:” As used herein or grammatical equivalents thereof, indicate values that are relative to a baseline measurement, such as a measurement in the same individual prior to initiation of a treatment described herein, or a measurement in a control individual (or multiple control individuals) in the absence of the treatment described herein. In some embodiments, a “control individual” is an individual afflicted with the same form of disease or injury as an individual being treated.

“Microcoring:” As used herein, “microcoring” refers to technologies that utilize one or more (in some embodiments, a plurality, e.g., an array) hollow needles or other non-thermal implement of sufficiently small dimension to minimize the extent of bleeding and/or clotting within the holes or slits and/or to minimize scar formation to excise and optionally sequester tissue from a site.

“Patient:” As used herein, the term “patient” refers to any organism to which a provided composition is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is or includes cancer, or presence of one or more tumors. In some embodiments, the patient is receiving or has received certain therapy to diagnose and/or to treat a disease, disorder, or condition.

“Prevent” or “prevention:” As used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

“Response:” As used herein, a response to treatment may refer to any beneficial alteration in a subject's condition that occurs as a result of or correlates with treatment. Such alteration may include stabilization of the condition (e.g., prevention of deterioration that would have taken place in the absence of the treatment), amelioration of symptoms of the condition, and/or improvement in the prospects for cure of the condition, etc.

“Risk:” As will be understood from context, “risk” of a disease, disorder, and/or condition comprises likelihood that a particular individual will develop a disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

“Sequestering:” As used herein, “sequestering” means, when used in reference to tissue, excising a microcore and then removing the excised microcore from the excision site.

“Subject:” As used herein, “subject” means an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

“Substantially:” As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

“Therapeutic agent:” As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

“Treatment:” As used herein, the term “treatment” (also “treat” or “treating”) refers to any administration of a therapy that partially or completely alleviates, ameliorates, relives, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

### DETAILED DESCRIPTION OF CERTAIN EMBODIMENTS

Described herein are technologies, methods, and/or devices for treating skin (e.g., eliminating tissue volume, tightening skin, lifting skin, and/or reducing skin laxity) by selectively excising a plurality of microcores without thermal energy being imparted to the surrounding (e.g., non-excised) tissue. In certain embodiments, the excising is completed within a certain time period, or is performed at a certain rate. In certain embodiments, the treatment is performed in specific areas not treatable with certain thermal methods, e.g., in the vicinity of nerves and/or other heat sensitive areas. In certain embodiments, a cosmetic effect is visible during treatment, immediately after or within a very short time after completion of treatment. In certain embodiments, a cosmetic effect is visible 1 min, 5 min, 10 min, 20 min, 30 min, 1 hour, 2 hours, 3 hours, 6 hours, 12 hours, 24 hours, 2 days, 3 days, 4 days, 5 days, 6 days, 7 days, 2 weeks, 3 weeks, 1 month, 2 months, 3 months, 4 months, 5 months, or 6 months after completion of treatment.

### Microcoring

In general, the term “microcoring,” as used herein, refers to technologies that utilize one or more (in some embodiments, a plurality, e.g., an array) hollow needles or other non-thermal implement of sufficiently small dimension to minimize the extent of bleeding and/or clotting within the holes or slits and/or to minimize scar formation to excise and optionally sequester tissue from a site. In some embodiments, excising a tissue means forming a tissue portion (the “microcore”), e.g., by inserting a hollow needle into the site so that the tissue portion is formed inside the hollow needle and severed from surrounding tissue so that a microcore that is separate from other tissue is generated.

Moreover, microcoring technologies as described herein can include sequestration of the excised tissue. As used herein, the term “sequestering”, when used in reference to tissue, means excising a microcore and then removing the excised microcore from the excision site. In certain embodiments, sequestered tissue is permanently disposed. In certain embodiments, sequestered tissue is used for diagnostic purpose, e.g., using biopsy and/or histology techniques known in the art. In many embodiments, technologies provided herein maximize removal and minimize risk of (partial or complete) re-insertion of extracted tissue.

It should be understood that microcoring technologies, methods, and/or devices using hollow needles described herein serve for exemplary and/or illustrative purposes, and that other techniques and devices can be used to create microcores. Representative such microcoring techniques and devices are described, for example, in U.S. patent application Ser. No. 14/910,767, filed Feb. 8, 2016, and/or Provisional Patent Application No. 62/314,748, filed Mar. 29, 2016, both of which are incorporated herein by reference in their entireties.

Microcoring technologies described herein a number of advantageous features.

For example, provided technologies may enable visualization of results in real time during the course of the treatment, e.g., through patient feedback and subsequent treatment adjustment in real time.

Alternatively or additionally, apparatuses used for microcoring can include micro-sized features that can be beneficial for controlling extent of skin treatment.

Still further, in some embodiments, methods and/or devices described herein may require less skill than that of a surgeon. Thus, in certain embodiments, patients may be treated in an outpatient setting, rather than in an inpatient, surgical setting. In some embodiments, subjects may be treated at a spa, at a cosmetic salon, or at home. That is, the present disclosure provides technologies that are amenable to and/or permit consistent and/or reproducible administration of skin treatment services.

In some embodiments, technologies, methods, and/or devices described herein have generally a lower risk profile and can provide more predictable results and/or risk factors than those for more invasive techniques (e.g., plastic surgery) or noninvasive energy-based techniques (e.g., laser, radiofrequency (RF), or ultrasound). In some embodiments, non-thermal fractional excision technologies, methods, and/or devices described herein allow skin tightening, skin lifting, and/or reduction of skin laxity without (or with significant reduction of) one or more common side effects of thermal ablation methods. Thermal ablation techniques prevent and/or inhibit skin tightening by allowing coagulation of tissue and formation of rigid tissue cores that cannot be compressed. Thermal ablation techniques create a three-dimensional heat-affected zone (HAZ) surrounding an immediate treatment site. While fractional ablative lasers can be used on or near heat-sensitive sites (e.g., eyes, nerves), i.e., when the laser does not penetrate more than 1 mm into the skin (resulting in a comparatively small HAZ), other thermal ablation techniques (e.g., ultrasound based techniques) cannot be used in the vicinity of heat-sensitive sites because the HAZ may extend to heat sensitive tissues potentially causing (permanent) damage. As will be appreciated by those skilled in the art reading the present disclosure, a “heat-sensitive site” is a site where exposure to radiation and/or elevated temperature is associated with a relatively high risk of unacceptable cosmetic and/or physiologic outcomes. In any event, technologies, methods, and/or devices described herein have generally a lower risk profile at least in part due to a zone of tissue injury that is smaller than the zone of injury (e.g., the HAZ) of thermal methods.

In some embodiments, advantages of certain technologies, methods, and/or devices described herein include a lesser degree of erythema, faster resolution of erythema, and lower percent incidence, severity, term of skin discoloration (hyperpigmentation or hypopigmentation), and/or less swelling and/or inflammation, as compared, for example, with that observed with laser treatment and/or with ultrasound-based treatment.

In some embodiments, certain technologies, methods, and/or devices provided herein can allow for rapid closing of holes or slits after excising tissue (e.g., within a few seconds after treating skin, such as within ten seconds), thereby minimizing extent of bleeding and/or clotting within holes or slits, and/or scar formation.

In some embodiments, certain technologies, methods, and/or devices provided herein can be useful for maximizing treatment effect while minimizing treatment time, e.g., by using rapid-fire reciprocating needles or needle arrays, and/or by using large needle arrays that allow for simultaneous excision of tens, hundreds, or even thousands of microcores.

In some embodiments, technologies, methods, and/or devices described herein can be useful for maximizing tightening effect while minimizing healing time and/or minimizing the time in which a cosmetic effect occurs by optimizing tightening (e.g., by controlling the extent of skin pleating, such as by increasing the extent of skin pleating for some applications or skin regions and by decreasing the extent of skin pleating for other applications or skin regions, as described herein).

In some embodiments, technologies, methods, and/or devices described herein can provide efficient clearance of sequestered or partially ablated tissue and/or debris from ablated tissue portions, thus reducing time for healing and improving the skin tightening treatment, e.g., relative to laser-based technologies.

In some embodiments, technologies, methods, and/or devices described herein can allow for efficient and effective positioning of skin prior to, during, and after excision and/or tissue sequestration. Positioning the skin is critical to control skin-tightening direction and ensure ablation occurs in the desired location and desired dimensions (e.g. thickness, width in a preferred direction, e.g., along or orthogonal to Langer lines).

Among other things, the present disclosure encompasses the insight that microcoring technologies can be developed (e.g., as described herein) that can achieve desirable procedure times and/or can significantly improve one or more aspects of healing from a procedure (e.g., a tissue removal procedure), compared to, e.g., thermal methods.

### Procedures

In some embodiments, technologies, methods, and/or devices described herein may be used for cosmetic resurfacing of skin tissue by removing skin tissue portions.

Technologies, methods, and/or devices described herein can be applied to treat one or more skin regions. In particular embodiments, these regions are treated with one or more procedures to improve skin appearance and/or to rejuvenate skin. In certain embodiments, technologies, methods, and/or devices described herein can be useful for skin tightening, e.g., reducing skin laxity (e.g., loose or sagging skin, or other skin irregularities).

In certain embodiments, technologies, methods, and/or devices described herein can be useful for removal of, e.g., redundant or excess skin, pigment, hair follicles, and/or vessels in the skin, and/or for treating acne, allodynia, blemishes, ectopic dermatitis, hyperpigmentation, hyperplasia (e.g., lentigo or keratosis), loss of translucency, loss of elasticity, melasma (e.g., epidermal, dermal, or mixed subtypes), photodamage, rashes (e.g., erythematous, macular, papular, and/or bullous conditions), psoriasis, rhytides (or wrinkles, e.g., lateral canthal lines (“crow's feet”), age-related rhytides, sun-related rhytides, or heredity-related rhytides), sallow color, scar contracture (e.g., relaxation of scar tissue), scarring (e.g., due to acne, surgery, or other trauma), skin aging, skin contraction (e.g., excessive tension in the skin), skin irritation/sensitivity, striae (or stretch marks), tattoo removal, vascular lesions (e.g., angioma, erythema, hemangioma, papule, port wine stain, rosacea, reticular vein, or telangiectasia), or any other unwanted skin irregularities. The technologies, methods, and/or devices described herein may also be used to penetrate skin and trigger biological responses that may contribute to new skin tissue formation and tissue resurfacing and remodeling.

In certain embodiments, technologies, methods, and/or devices described herein can be applied to a site located on any part or parts of the body, including face (e.g., eyelid, cheeks, chin, forehead, lips, or nose), neck, chest (e.g., as in a breast lift), arms, hands, legs, abdomen, buttock, and thigh. In certain specific embodiments, a treatment site is located on the face and/or neck. In certain embodiments, a site is located on a part of the body that is heat sensitive. Such a site is generally not amenable to treatment with deep-penetrating thermal methods. For example, lasers can only be used if laser energy is targeted to the epidermis/dermis only and thus creates only a comparatively small HAZ. In certain embodiments, a site is located on the face in close proximity to an eye. In certain embodiments, a site is located on the face in close proximity to the facial nerve or a facial nerve branch, e.g. the temporal branch, the zygomatic branch, the buccal branch, the marginal mandibular branch, or the cervical branch.

In certain embodiments, a site is located over or near the thyroid gland, thyroid cartilage, trachea, a major blood vessel, or breast tissue.

In certain embodiments, a treatment site can have any size. In certain embodiments, a treatment site has an area of between 1 cm² and 10,000 cm², between 10 cm² and 5,000 cm², between 100 cm² and 2,500 cm², or between 500 cm² and 1,000 cm². In certain embodiments, the treatment site has an area of between 1 cm² and 300 cm², between 1.2 cm² and 280 cm², between 1.4 cm² and 260 cm², between 1.6 cm² and 240 cm², between 1.8 cm² and 220 cm², between 2 cm² and 200 cm², between 2.2 cm² and 180 cm², between 2.4 cm² and 160 cm², between 2.6 cm² and 140 cm², between 2.8 cm² and 120 cm², between 3 cm² and 100 cm², between 3.2 cm² and 80 cm², between 3.4 cm² and 60 cm², between 3.6 cm² and 40 cm², between 3.8 cm² and 20 cm², between 4 cm² and 10 cm², between 10 cm² and 20 cm², between 20 cm² and 30 cm², between 30 cm² and 40 cm², between 40 cm² and 50 cm², between 50 cm² and 60 cm², between 60 cm² and 70 cm², between 70 cm² and 80 cm², between 80 cm² and 100 cm², between 100 cm² and 120 cm², between 120 cm² and 140 cm², between 140 cm² and 160 cm², between 160 cm² and 180 cm², or between 180 cm² and 200 cm².

In certain embodiments, technologies, methods, and/or devices described herein may involve forming a plurality of holes in the skin, e.g., by contacting one or more hollow needles to the skin of a subject and excising or sequestering cored tissue portions from the skin. Penetration into the skin by, e.g., hollow needle(s), creates holes and so effectively reduces tissue volume and/or improves tissue quality upon healing. For example, forming a series of cored tissue portions (e.g., excising or sequestering about 20% of the total skin area) and corresponding holes in a high laxity skin region, and optionally subsequent compression of the skin region to close the holes may promote the growth of improved tissue (e.g., new skin). In other embodiments, treatment methods described herein can be used to reduce laxity or the appearance of laxity in the skin. In some embodiments, treatment methods described herein can be used to remove excess/redundant skin.

In certain embodiments, technologies, methods, and/or devices described herein further comprise application of a dressing. Healing of tissue under a dressing (e.g., a compressive or occlusive dressing) allows for the existing tissue to span the gap introduced by the removal of cored tissue portions, thereby reducing skin volume and area (e.g., by tightening the skin). In some embodiments, application of a dressing (e.g., a compressive or occlusive dressing) may help to maintain moisture of a treated skin area and/or to prevent delivered therapeutic agents from leaking out of the skin. In some embodiments, application of a dressing may improve the healing profile of the treated region, e.g., by providing hemostatic pressure to cored regions.

Microcoring can be performed by creating holes in the skin at various hole densities. In certain embodiments, tissue can be excised or sequestered from the treatment region with various hole densities (e.g., the number of holes per unit area) corresponding to the number and geometry of hollow needle(s) of the apparatus used and the number of applications of the hollow needle(s) to the treatment region. Different hole densities may be desirable for different regions of skin and for different conditions, and may be achieved using different hollow needle(s). For example, 15 holes corresponding to the size of a 19 gauge needle and their corresponding cored tissue portions may be created in a given treatment area by actuation of a single 19 gauge needle 15 times, or by actuating an array having five 19 gauge needles three times. Spacing the same number of holes further apart will result in a lower hole density per unit area. For example, 15 holes may be created within a 0.5 mm by 0.3 mm region or within a 5 mm by 3 mm region. In certain embodiments, technologies, methods, and/or devices described herein are configured to provide from about 10 to about 10000 cored tissue portions per cm² area of a site (e.g., as described herein). An array of holes created by removal of skin tissue portions may be created in any beneficial pattern within a site. For example, a higher density and/or smaller spacing of tissue portions and corresponding holes can be excised or sequestered in skin in the center of a pattern or in thicker portions of skin. A pattern may be semi-random or include one or more of staggered rows and/or blocks, parallel rows and/or blocks, a circular pattern, a spiral pattern, a square or rectangular pattern, a triangular pattern, a hexagonal pattern, a radial distribution, or a combination of one or more such patterns. A pattern may arise from the use of one or more hollow needles (or other microcoring implements) with one or more configurations and numbers of hollow needles (or other microcoring implements) applied in any ordered or disordered manner. Modifications to the average length, diameter, shapes, and/or other characteristics of one or more hollow needles (or other microcoring implements) used to treat a skin region may also result in a specific pattern of holes in the skin. Such patterns may be optimized to promote unidirectional, non-directional, or multidirectional contraction or expansion of skin (e.g., in the x-direction, y-direction, x-direction, x-y plane, y-z plane, x-z plane, and/or xyz-plane), such as by modifying the average length, depth, diameter, density, orientation, and/or spacing between hollow needles. Additionally, the orientation of a needle can provide the basis for an oriented pattern. For example, proximal ends of needles can be non-uniform, and, e.g., may comprise one or more prongs. Insertion of needle tips comprising prongs into the skin can lead to cores that are not perfectly cylindrical throughout the extent of the core. To the extent that the cores are not cylindrical, patters may be produced in treated skin, e.g., by controlling the relative positions of the prongs during a coring process. For example, prongs may be held at a fixed angle relative to the x-y pattern of strikes, or the prong positions may be alternated to produce complex patterns.

Micorcores can have any diameter. In certain embodiments, a diameter largely corresponds to the inner diameter of hollow needles described herein. In certain embodiments, the microcores may have a diameter of between about 0.1 mm and about 1.0 mm, or between 0.14 mm and about 0.84 mm, 0.16 mm and about 0.82 mm, 0.18 mm and about 0.8 mm, 0.2 mm and about 0.78 mm, 0.22 mm and about 0.76 mm, 0.24 mm and about 0.74 mm, 0.26 mm and about 0.72 mm, 0.28 mm and about 0.7 mm, 0.3 mm and about 0.68 mm, 0.32 mm and about 0.66 mm, 0.34 mm and about 0.64 mm, 0.36 mm and about 0.62 mm, 0.38 mm and about 0.6 mm, 0.4 mm and about 0.58 mm, 0.42 mm and about 0.56 mm, 0.44 mm and about 0.54 mm, 0.46 mm and about 0.52 mm, or 0.48 mm and about 0.5 mm, (e.g., 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 5 0.74, 0.75, 0.76, 0.77, 0.78, 0.79, 0.8, 0.81, 0.82, 0.83, and 0.84 mm). In certain embodiments, microcores may have a diameter of between about 0.1 mm and about 1.0 mm, about 0.14 mm and about 0.84 mm, about 0.24 mm and about 0.40 mm (e.g., 0.24, 0.25, 0.26, 0.27, 0.28, 0.29, 0.3, 0.31, 0.32, 0.33, 0.34, 0.35, 0.36, 0.37, 0.38, 0.39, and 0.4 mm). In a certain embodiments, microcores may have a diameter of from about 0.25 mm to about 0.6 mm. In certain embodiments, depending on the microcoring technique used, the diameter of each microcore varies along the length of the microcore. The shape of a microcore may be cylindrical, conical, hemispherical, hyperboloid or any combination thereof, or any other shape.

Thickness of skin can vary significantly between different regions of a body.

Skin in the periphery of an eye and an eyelid are very thin, while skin at or near the sole of a foot is very thick. In addition, scar tissue can be even thicker than normal skin. Any portion of skin can be excised using the technologies, methods, and/or devices described herein. Tissue portions created by microcoring may include epidermal tissue, dermal tissue, subcutaneous fat, and/or cells or tissue proximal to the dermal/fatty layer boundary (e.g., stem cells). In certain embodiments, a tissue portion may have a length that corresponds to depth of penetration of the skin layer with a microneedle. In certain embodiments, depth of penetration may be (i) into the dermal layer, (ii) through the entire dermal layer to the junction of a dermal layer and the subcutaneous fat layer, or (iii) into the subcutaneous fat layer. Total depth of epidermal, dermal, and subcutaneous fat layers may vary based on the region and age of the body being treated. In some instances, depth of the epidermal layer is between about 0.01 mm to 0.2 mm, and/or depth of the dermal layer is between about 0.3 mm to 6.0 mm. In some embodiments, total depth of the epidermal and dermal layers may be between about 0.3 mm and 6.2 mm, corresponding to a possible tissue portion having a length of between about 0.3 mm and 6.2 mm (e.g., between about 0.3 mm and 0.6 mm, 0.3 mm and 0.9 mm, 0.3 mm and 1.5 mm, 0.3 mm and 2.0 mm, 0.3 mm and 2.5 mm, 0.3 mm and 3.0 mm, 0.3 mm and 3.5 mm, 0.3 mm and 4.0 mm, 0.3 mm and 4.5 mm, 0.3 mm and 5.0 mm, 0.3 mm and 5.5 mm, 0.3 mm and 6.0 mm, 0.3 mm and 6.2 mm, 0.6 mm and 0.9 mm, 0.6 mm and 1.5 mm, 0.6 mm and 2.0 mm, 0.6 mm and 2.5 mm, 0.6 mm and 3.0 mm, 0.6 mm and 3.5 mm, 0.6 mm and 4.0 mm, 0.6 mm and 4.5 mm, 0.6 mm and 5.0 mm, 0.6 mm and 5.5 mm, 0.6 mm and 6.0 mm, 0.6 mm and 6.2 mm, 0.9 mm and 1.5 mm, 0.9 mm and 2.0 mm, 0.9 mm and 2.5 mm, 0.9 mm and 3.0 mm, 0.9 mm and 3.5 mm, 0.9 mm and 4.0 mm, 0.9 mm and 4.5 mm, 0.9 mm and 5.0 mm, 0.9 mm and 5.5 mm, 0.9 mm and 6.0 mm, 0.9 mm and 6.2 mm, 1.5 mm and 2.0 mm, 1.5 mm and 2.5, mm, 1.5 mm and 3.0 mm, 1.5 mm and 3.5 mm, 1.5 mm and 4.0 mm, 1.5 mm and 4.5 mm, 1.5 mm and 5.0 mm, 1.5 mm and 5.5 mm, 1.5 mm and 6.0 mm, 1.5 mm and 6.2 mm, 2.0 mm and 2.5 mm, 2.0 mm and 3.0 mm, 2.0 mm and 3.5 mm, 2.0 mm and 4.0 mm, 2.0 mm and 4.5 mm, 2.0 mm and 5.0 mm, 2.0 mm and 5.5 mm, 2.0 and 6.0 mm, 2.0 mm and 6.2 mm, 2.5 mm and 3.0 mm, 2.5 mm and 3.5 mm, 2.5 mm and 4.0 mm, 2.5 mm and 4.5 mm, 2.5 mm and 5.0 mm, 2.5 mm and 5.5 mm, 2.5 mm and 6.0 mm, 2.5 mm and 6.2 mm, 3.0 mm and 3.5 mm, 3.0 mm and 4.0 mm, 3.0 mm and 4.5 mm, 3.0 mm and 5.0 mm, 3.0 mm and 5.5 mm, 3.0 and 6.0 mm, 3.0 mm and 6.2 mm, 3.5 mm and 4.0 mm, 3.5 mm and 4.5 mm, 3.5 mm and 5.0 mm, 3.5 mm and 5.5 mm, 3.5 and 6.0 mm, 3.5 mm and 6.2 mm, 4.0 mm and 4.5 mm, 4.0 mm and 5.0 mm, 4.0 mm and 5.5 mm, 4.0 and 6.0 mm, 4.0 mm and 6.2 5 mm, 4.5 mm and 5.0 mm, 4.5 mm and 5.5 mm, 4.5 and 6.0 mm, 4.5 mm and 6.2 mm, 5.0 mm and 5.5 mm, 5.0 mm and 6.0 mm, 5.0 mm and 6.2 mm, 5.5 mm and 6.0 mm, 5.5 mm and 6.2 mm, or 6.0 mm and 6.2 mm). In certain embodiments, the possible tissue portion has a length between about 0.3 and 2 mm, or between about 0.3 and 3 mm.

In certain embodiments, excised tissue portions extend into the subcutaneous fat layer. In certain embodiments, a tissue portions created by microcoring can extend from the junction of the dermal layer and the subcutaneous fat layer into the subcutaneous fat layer by between about 0.1 mm and 3 mm, 0.2 mm and 2.8 mm, 0.3 mm and 2.6 mm, 0.4 mm and 2.4 mm, 0.5 mm and 2.2 mm, 0.6 mm and 2 mm, 0.8 mm and 1.8 mm, or 1 mm and 1.6 mm. Thus, a possible tissue portion can have a length between about 6.2 mm and 9.2 mm, 6.2 mm and 9 mm, 6.2 mm and 8.8 mm, 6.2 mm and 8.6 mm, 6.2 mm and 8.4 mm, 6.2 mm and 8.2 mm, 6.2 mm and 8 mm, 6.2 mm and 7.8 mm, 6.2 mm and 7.6 mm, 6.2 mm and 7.4 mm, 6.2 mm and 7.2 mm, 6.2 mm and 7 mm, 6.2 mm and 6.8 mm, 6.2 mm and 6.6 mm, or 6.2 mm and 6.4 mm.

In certain embodiments, tissue portions are excised from scar tissue. In certain embodiments, an excised tissue portion can extend through the scar/skin tissue layers into the subcutaneous fat layer corresponding to a possible tissue portion having a length between about 0.1 mm and 20 mm, 0.2 mm and 18 mm, 0.3 mm and 16 mm, 0.4 mm and 14 mm, 0.5 mm and 12 mm, 0.6 mm and 10 mm, 0.8 mm and 8 mm, 1 mm and 6 mm, 1.2 mm and 4 mm, or 1.4 mm and 2 mm.

In certain embodiments, technologies, methods, and/or devices described herein further include technologies to ensure consistent depth of penetration and orientation, e.g., depth of penetration and orientation of hollow needles, into skin. In certain embodiments, technologies, methods, and/or devices described herein can be configured to accommodate different skin thicknesses. In certain embodiments, devices, e.g., devices comprising reciprocating needles, can include mechanical technologies (e.g., spacers) to ensure constant distance between a needle tip and skin when the needle is fully retracted, and technologies for adjustment of the distance the needle travels along its longitudinal axis on each actuation.

Micorcores can have any volume. In certain embodiments, volume largely corresponds to the cross-sectional area of a hollow needles described herein multiplied by a length of an excised tissue portion. In certain embodiments, microcores may have a volume of between about 0.001 mm³ and 6.3 mm³, 0.005 mm³ and 5.0 mm³, 0.01 mm³ and 4.0 mm³, 0.015 mm³ and 3.0 mm³, 0.02 mm³ and 2.0 mm³, 0.022 mm³ and 1.8 mm³, 0.024 mm³ and 1.6 mm³, 0.026 mm³ and 1.4 mm³, 0.028 mm³ and 1.2 mm³, 0.03 mm³ and 1.0 mm³, 0.032 mm³ and 0.8 mm^{3, 0.034} mm³ and 0.6 mm³, 0.036 mm³ and 0.4 mm³, 0.038 mm³ and 0.2 mm³, 0.04 mm³ and 0.1 mm³, or 0.06 mm³ and 0.08 mm³.

In certain embodiments, technologies, methods, and/or devices described herein comprise microcoring a tissue below an epidermis layer only, leaving the epidermis, or other tissue layer above the layer to be excised, un-cored (i.e., no tissue is excised from the epidermis other tissue layer above the layer to be excised). In certain embodiments, a microcoring implement, e.g., a needle, has a first configuration that allows it to travel through a tissue layer with minimal damage to said tissue layer, e.g., by traveling through tissue like a solid needle, and has a second configuration that allows for the formation of a microcore, e.g., by traveling through tissue like a hollow needle. In certain embodiments, a device undergoes a change in configuration from a first configuration to a second configuration as it transitions longitudinally from a first tissue layer into a second tissue layer. In certain embodiments, the device undergoes a change in configuration from a first configuration to a second configuration as it transitions longitudinally from an epidermis into a dermis. In certain embodiments, a device undergoes a change in configuration from a first configuration to a second configuration as it transitions longitudinally from the dermis into the subcutaneous fat layer. In certain embodiments, a process of microcoring a tissue below an epidermis layer only, e.g., leaving the epidermis, or other tissue layer above the layer to be excised, un-cored occurs independently of treatment time and/or treatment speed.

Technologies, methods, and/or devices described herein can employ microcoring implements, e.g., hollow needles, that are arranged in any configuration. In certain embodiments, microcoring implements, e.g., hollow needles, are mounted on a reciprocating device configured to (i) translate the needles in a direction substantially along the longitudinal axis of the needles and/or (ii) translate the needles over the skin tissue in one or two orthogonal directions. In certain embodiments, a reciprocating device may have as few as 1 or as many as hundreds of hollow needles. In certain embodiments, 1-100 hollow needles may be present (e.g., 1-10, 1-20, 1-30, 1-40, 1-50, 1-60, 1-70, 1-80, 1-90, 1-100, 3-10, 3-20, 3-30, 3-40, 3-50, 3-60, 3-70, 3-80, 3-90, 3-100, 5-10, 5-20, 5-30, 5-40, 5-50, 5-60, 5-70, 5-80, 5-90, 5-100, 10-20, 10-40, 10-60, 10-80, 10-100, 20-40, 20-60, 20-80, 20-100, 40-60, 40-80, 40-100, 60-80, 60-100, or 80-100 hollow needles). Use of an array of a plurality of hollow needles to generate an array pattern may facilitate skin treatment over larger areas and in less time.

In certain embodiments, needles are mounted on a non-reciprocating application device (an “applique”) in form of an array, e.g., and as described above. In certain embodiments, an applique, and thus each needle contained therein, is applied to the site only once. In certain embodiments, an applique, and thus each needle contained therein, is applied to a site 2, 3, 4, or more times. In certain embodiments, application of an applique is independent of treatment time and/or treatment speed, e.g., application of an applique can occur instantaneously. In certain embodiments, application of an applique occurs in less than about 1 second. In certain embodiments, an applique/array comprises at least 10 needles, at least 100 needles, at least 1000 needles, at least 10,000 needles, or at least 100,000 needles. In certain embodiments, an array comprises between 10 and 100,000 needles, between 20 and 50,000 needles, between 30 and 25,000 needles, between 40 and 15,000 needles, between 50 and 10,000 needles, between 60 and 8,000 needles, between 70 and 6,000 needles, between 80, and 4,000 needles, between 90 and 2,000 needles, between 100 and 1,000 needles, between 120 and 800 needles, between 140 and 600 needles, or between 160 and 400 needles.

Technologies, methods, and/or devices described herein can comprise one or more microcoring implements, e.g., hollow needles, that may be configured to provide from about 10 to about 10000 cored tissue portions, or more, per cm² area (e.g., 10 to 50, 10 to 100, 10 to 200, 10 to 300, 10 to 400, 10 to 500, 10 to 600, 10 to 700, 10 to 800, 10 to 900, 10 to 1000, 10 to 2000, 10 to 4000, 10 to 6000, 10 to 8000, 10 to 10000, 50 to 100, 50 to 200, 50 to 300, 50 to 400, 50 to 500, 50 to 600, 50 to 700, 50 to 800, 50 to 900, 50 to 1000, 50 to 2000, 50 to 4000, 510 to 6000, 50 to 8000, 50 to 10000, 100 to 200, 100 to 300, 100 to 400, 100 to 500, 100 to 600, 100 to 700, 100 to 800, 100 to 900, 100 to 1000, 100 to 2000, 100 to 4000, 100 to 6000, 100 to 8000, 100 to 10000, 200 to 300, 200 to 400, 200 to 500, 200 to 600, 200 to 700, 200 to 800, 200 to 900, 200 to 1000, 200 to 2000, 200 to 4000, 200 to 6000, 200 to 8000, 200 to 10000, 300 to 400, 300 to 500, 300 to 600, 300 to 700, 300 to 800, 300 to 900, 300 to 1000, 300 to 2000, 300 to 4000, 300 to 6000, 300 to 8000, 300 to 10000, 400 to 500, 400 to 600, 400 to 700, 400 to 800, 400 to 900, 400 to 1000, 400 to 2000, 400 to 4000, 400 to 6000, 400 to 8000, 400 to 10000, 500 to 600, 500 to 700, 500 to 800, 500 to 900, 500 to 1000, 500 to 2000, 500 5 to 4000, 500 to 6000, 500 to 8000, 500 to 10000, 600 to 700, 600 to 800, 600 to 900, 600 to 1000, 600 to 2000, 600 to 4000, 600 to 6000, 600 to 8000, 600 to 10000, 700 to 800, 700 to 900, 700 to 1000, 700 to 2000, 700 to 4000, 700 to 6000, 700 to 8000, 700 to 10000, 800 to 900, 800 to 1000, 800 to 2000, 800 to 4000, 800 to 6000, 800 to 8000, 800 to 10000, 900 to 1000, 900 to 2000, 900 to 4000, 900 to 6000, 900 to 8000, 900 to 10000, 1000 to 2000, 1000 to 4000, 1000 to 6000, 1000 to 8000, 1000 to 10000, 2000 to 4000, 2000 to 6000, 2000 to 8000, 2000 to 10000, 4000 to 6000, 4000 to 8000, 4000 to 10000, 6000 to 8000, 6000 to 10000, and 8000 to 10000 tissue portions per cm² area) of a skin region to which the apparatus is applied (e.g., a site or treatment area).

Any beneficial area or volumetric fraction of a skin region can be removed. For example, between about 1% to about 65% (e.g., an areal fraction between about 0.01 to about 0.65, such as 0.01 to 0.65, 0.01 to 0.6, 0.01 to 0.55, 0.01 to 0.5, 0.01 to 0.45, 0.01 to 0.4, 0.01 to 0.35, 0.01 to 0.3, 0.01 to 0.25, 0.01 to 0.2, 0.01 to 0.15, 0.01 to 0.1, 0.01 to 0.05, 0.03 to 0.65, 0.05 to 0.65, 0.07 to 0.65, 0.09 to 0.65, 0.1 to 0.65, 0.15 to 0.65, 0.2 to 0.65, 0.25 to 0.65, 0.3 to 0.65, 0.35 to 0.65, 0.4 to 0.65, 0.45 to 0.65, 0.5 to 0.65, 0.55 to 0.65, and 0.6 to 0.65) of tissue in a treatment area or site may be removed. In certain embodiments, between about 1% to about 5% (e.g., an areal fraction between about 0.01 to about 0.05, such as 0.01 to 0.05, 0.01 to 0.045, 0.01 to 0.04, 0.01 to 0.035, 0.01 to 0.03, 0.01 to 0.025, 0.01 to 0.02, 0.01 to 0.015, 0.015 to 0.05, 0.02 to 0.05, 0.025 to 0.05, 0.03 to 0.05, 0.035 to 0.05, 0.04 to 0.05, and 0.045 to 0.05) of tissue in a treatment area or site may be removed. In certain embodiments, between about 2% to about 3% (e.g., an areal fraction between about 0.02 to about 0.03, such as 0.02 to 0.03, 0.02 to 0.028, 0.02 to 0.026, 0.02 to 0.024, 0.02 to 0.022, 0.022 to 0.03, 0.024 to 0.03, 0.026 to 0.03, 0.028 to 0.03; e.g., 0.025) of tissue in a treatment area or site may be removed.

In some embodiments, skin may be elevated, compressed and/or stretched immediately prior to and/or during microcoring. In certain embodiments, technologies, devices and/or methods described herein comprise positioning skin using a compressive and/or a stretching force applied across the skin prior to or during microcoring. In certain embodiments, technologies, devices and/or methods comprise a positioning apparatus for positioning skin, said apparatus comprising, e.g., at least two sufficiently parallel tensioning rods configured to elevate, compress and/or stretch skin, or a plurality of microhooks or microbarbs configured to elevate, compress and/or stretch skin, or a vacuum source configured to elevate, compress and/or stretch skin.

Technologies, methods, and/or devices described herein further comprise methods for procedure preparation, e.g., skin preparation and/or device preparation, prior to initiation of a microcoring procedure. Microcoring procedures take place under aseptic conditions. Exemplary preparation methods include cleaning a site with alcohol (e.g., ethanol) prior to commencement of treatment. In certain embodiments, the technologies, methods, and/or devices further comprise inducing local anesthesia/analgesia. In certain embodiments, a local anesthetic (e.g., lidocaine, bupivacaine, or a sodium channel blocker) are injected into skin at and/or near a site. In certain specific embodiments, epinephrine is injected before, after, or simultaneously with a local anesthetic. Without wishing to be bound by theory, epinephrine acts as a vasoconstrictor and thus slows the absorption of a local anesthetic, thus prolonging the action of the anesthetic. In addition, epinephrine reduces bleeding during a microcoring procedure. In certain embodiments, a local anesthetic, e.g., lidocaine, and epinephrine are administered together diluted in a saline solution. In certain other embodiments, a local anesthetic, e.g., lidocaine, is used for nerve block and is injected in a small quantity near a nerve of interest. In certain other embodiments, a local anesthetic, e.g., lidocaine, is used in a tumescent lidocaine/epinephrine regimen. In certain other embodiments, a topical anesthetic, e.g., lidocaine, can be applied prior to commencement of a micorcoring procedure. In certain specific embodiments, a topical anesthetic is applied 30 minutes to 60 minutes prior to commencement of a microcoring procedure. In certain other embodiments, pain may be modulated by lowering the temperature at a skin surface, e.g., by contacting skin with a cold surface or by blowing cold air over a skin surface prior to, during, or after microcoring. In certain embodiments, total preparation time to commencement of the microcoring procedure comprises cleaning, sterilizing, assembling, maintaining, and/or testing a microcoring device. In certain embodiments, total preparation time prior to commencement of the microcoring procedure may be less than 2 hours, less than 1 hour, less than 45 minutes, less than 30 minutes, less than 25 minutes, less than 20 minutes, less than 15 minutes, less than 10 minutes, less than 5 minutes, or less than 1 minute.

In some embodiments, technologies, methods, and/or devices described herein further comprise, utilize, or involve certain aspects of skin care after completion of a microcoring procedure. In certain embodiments, a sterile dressing, e.g., Vaseline is applied to a site. In certain embodiments, sterile Vaseline is applied immediately after completion of a microcoring procedure and/or for a period of one or more weeks thereafter. Without wishing to be bound by theory, a sterile dressing limits the risk of infection by creating a barrier to infectious agents, and maintains moisture of the skin. In certain embodiments, additional medication may be administered either locally or systemically. In certain other embodiments, healing may be accelerated by lowering the temperature at a skin surface, e.g., by contacting skin with a cold surface or by blowing cold air over a skin surface prior to, during, or after microcoring. In certain embodiments, total aftercare time immediately after completion of a microcoring procedure may be less than 2 hours, less than 1 hour, less than 45 minutes, less than 30 minutes, less than 25 minutes, less than 20 minutes, less than 15 minutes, less than 10 minutes, less than 5 minutes, or less than 1 minute. In certain embodiments, a patient having undergone a microcoring procedure may not require overnight stay at the treatment facility. In certain embodiments, a patient having undergone a microcoring procedure may be discharged from the treatment facility less than 2 hours, less than 1 hour, less than 45 minutes, less than 30 minutes, less than 25 minutes, less than 20 minutes, less than 15 minutes, less than 10 minutes, less than 5 minutes, or less than 1 minute after completion of treatment.

### Exemplary Devices

Representative microcoring techniques and devices are described, for example, in U.S. patent application Ser. No. 14/910,767, filed Feb. 8, 2016, and/or Provisional Patent Application No. 62/314,748, filed Mar. 29, 2016, both of which are incorporated herein by reference in their entireties.

Technologies, methods, and/or devices described herein comprise, utilize, or involve hollow needles, needle assemblies, actuation units, apparatuses, kits, and methods for cosmetic resurfacing of skin tissue by removing portions of the skin tissue. In some embodiments, technologies, methods, and/or devices described herein comprise an apparatus for generating a cosmetic effect in the skin tissue that includes one or more hollow needles each having at least one prong. In some embodiments, an apparatus may also include a mechanism for removing tissue portion(s) from the hollow needle(s).

In some embodiments, technologies, methods, and/or devices described herein comprise an apparatus for producing a cosmetic effect in a skin tissue that includes at least one hollow needle including at least a first prong provided at a distal end of the hollow needle, wherein an angle between a lateral side of the first prong and a longitudinal axis of the hollow needle is at least about 20 degrees, and wherein the hollow needle is configured to remove a portion of skin tissue when the hollow needle is inserted into and withdrawn from skin tissue.

In some embodiments, the angle between the lateral side of the first prong and the longitudinal axis of the hollow needle is between about 20 and about 40 degrees. In some embodiments, the angle between the lateral side of the first prong and the longitudinal axis of the hollow needle is about 30 degrees.

In some embodiments, a hollow needle further includes a second prong at the distal end of the hollow needle. In some embodiments, an angle between the lateral side of the second prong and the longitudinal axis of the hollow needle is at least about 20 degrees. In some embodiments, the angle between the lateral side of the second prong and the longitudinal axis of the hollow needle is between about 20 and about 40 degrees. In some embodiments, the lateral side of the second prong and the longitudinal axis of the hollow needle is about 30 degrees. In some embodiments, the angle between a lateral side of the second prong and a longitudinal axis of the hollow needle is less than about 20 degrees. In some embodiments, the angle between the lateral side of the second prong and the longitudinal axis of the hollow needle is between about 5 degrees and about 20 degrees.

In some embodiments, the first prong includes an edge. In some embodiments, each of the first and second prongs includes an edge.

In some embodiments, the first prong includes a flat tip. In some embodiments, each of the first and second prongs includes a flat tip. In some embodiments, the flat tip has a length and a width. In some embodiments, the length and/or the width is at an angle relative to the longitudinal axis of the hollow needle. In some embodiments, the length and/or the width is perpendicular to the longitudinal axis of the hollow needle.

The technologies, methods, and/or devices described herein comprise, utilize, or involve a needle assembly including a hollow needle, a z-actuator, and a tissue removal tool, wherein the hollow needle includes at least a first prong provided at a distal end of the hollow needle and wherein an angle (α) between a lateral side of the first prong and a longitudinal axis of the hollow needle is at least about 20 degrees.

In some embodiments, the hollow needle further includes a second prong. In some embodiments, an angle (α) between a lateral side of the second prong and a longitudinal axis of the hollow needle is at least about 20 degrees. In some embodiments, an angle (α) between a lateral side of the second prong and a longitudinal axis of the hollow needle is less than about 20 degrees.

In some embodiments, the first prong includes an edge. In some embodiments, each of the first and second prongs includes an edge. In some embodiments, the first prong includes a flat tip. In some embodiments, each of the first and second prongs includes a flat tip. In some embodiments, the flat tip has a length and a width. In some embodiments, the length and/or the width is at an angle relative to the longitudinal axis of the hollow needle. In some embodiments, the length and/or the width is perpendicular to the longitudinal axis of the hollow needle.

In some embodiments, a needle assembly further includes a support base, a scaffold, an aspiration tube, a trap, and/or a pressure generating source. In some embodiments, a needle assembly is configured to be detachably attached to an x- and/or y-actuator.

### Sites

As discussed above, technologies, methods, and/or devices described herein can be applied to treat specific sites or skin regions. In certain embodiments, technologies, methods, and/or devices described herein can be useful for skin tightening, e.g., reducing skin laxity (e.g., loose or sagging skin or other skin irregularities) in said sites.

In certain embodiments, technologies, methods, and/or devices described herein can be configured to treat any site. In certain embodiments, a site is characterized by its size and or location on a body as described above. In some embodiments, a site can be located where skin lacks a subcutaneous fat layer and/or is very thin, e.g., an eye lid, or where the skin comprises a subcutaneous fat layer, or where the skin comprises scar tissue, or a combination thereof.

In some embodiments, a site can be located where skin lacks or exhibits a low density of hair follicles, or exhibits a low to moderate presence of hair follicles, or exhibits a moderate to high presence of hair follicles, or exhibits a high to very high presence of hair follicles.

In certain embodiments, the site is characterized by a moderate to high presence of hair follicles.

In certain embodiments, a site can be located where skin exhibits a low perfusion or blood supply, or exhibits a low to moderate perfusion or blood supply, or exhibits a moderate to high perfusion or blood supply, or exhibits a high to very high perfusion or blood supply.

Without wishing to be bound by theory, sites with higher presence of hair follicles and/or blood supply are likely to heal better than sites with a lower presence of hair follicles and/or blood supply. A site can be on or near a major blood vessel. Without wishing to be bound by theory, major blood vessels are generally located at a tissue depth that is deeper than the depth of penetration of the technologies, methods, and/or devices described herein. In certain embodiments, technologies, methods, and/or devices described can be applied in a more aggressive manner on sites that heal better (e.g., on the face) than other sites. In certain embodiments, technologies, methods, and/or devices described can be applied in a more aggressive manner on sites that contain more skin (e.g., the abdomen) than other sites.

In certain embodiments, technologies, methods, and/or devices described herein further comprise separating the dermal layer from the superficial muscular aponeurotic system (SMAS) layer. In certain embodiments, technologies, methods, and/or devices described herein do not comprise separating the dermal layer from the SMAS layer. In certain embodiments, technologies, methods, and/or devices described herein further comprise removing the SMAS layer or a fraction of the SMAS layer.

In certain embodiments, a site is characterized by the inapplicability of certain thermal skin treatment methods. For example, certain thermal ablation techniques cannot be used in the vicinity of heat-sensitive sites (e.g., eyes, nerves) because their HAZ may extend to heat sensitive tissues potentially causing (permanent) damage. In certain embodiments, technologies, methods, and/or devices described herein can be used on or in the vicinity of a heat-sensitive site, e.g., an eye or a nerve.

In certain embodiments, technologies, methods, and/or devices described herein can be used on or in the vicinity of a nerve. In certain embodiments, technologies, methods, and/or devices comprise determining the presence of a nerve beneath a surface prior to removing/excising a microcore. In certain embodiments, technologies, methods, and/or devices described herein comprise determining the presence of a nerve via dynamic sensing. In certain embodiments, a sensor is integrated into a microcoring device or is a stand-alone unit. In certain embodiments, technologies, methods, and/or devices described herein comprise determining the presence of a nerve via detection using a feedback sensor, wherein the sensor detects transition from one tissue (e.g., dermal) layer to another. Without wishing to be bound by theory, in certain embodiments, a transition can be detected via measurement of the change of electric properties of a tissue surrounding the tip of a microcoring needle. In certain embodiments, technologies, methods, and/or devices described herein comprise determining presence of a nerve, for example via detection using nerve excitation. Without wishing to be bound by theory, in certain embodiments, presence of a nerve may be detected by inducing a small electric current from the tip of a microcoring needle, which excites a nerve in the surrounding tissue, resulting in a noticeable twitch. In certain embodiments, technologies, methods, and/or devices described herein comprise treating a site that is located on the face in close proximity to the facial nerve or a facial nerve branch. In certain embodiments, the site is located on the face in close proximity to the temporal branch, the zygomatic branch, the buccal branch, the marginal mandibular branch, or the cervical branch.

In certain embodiments, technologies, methods, and/or devices described herein comprise treating a site that is located over an area that comprises a mechanical implant, a dermal filler, or a breast implant. Mechanical implants containing metal may absorb and conduct heat such that the implant causes burns in the surrounding tissue. Certain thermal methods may reduce the effect of dermal fillers. Similarly, the materials in breast implants, e.g., silicone, may undergo undesired physical changes or impact the surrounding tissue when subjected to the level of heat generated by certain thermal skin treatment methods.

For example, certain thermal skin treatment methods are based on the application of high energy ultrasound waves to a tissue. The thermal response varies from tissue to tissue, largely based on the water content of each tissue type. Thus, each tissue type exhibits a different and often unpredictable, response to high energy ultrasound, e.g., due to each tissue type reflecting, refracting, or absorbing ultrasound waves differently. Such tissues include the thyroid gland, thyroid cartilage, trachea, a major blood vessel, or breast tissue. Thus, certain thermal methods, such as ultrasound based methods, require the use of medical imaging techniques before or during each procedure to ensure only the desired tissue portion is being treated where the application of high energy ultrasound is considered or know to be safe. In many tissue types or sites, the response of the underlying tissue to high intensity ultrasound has not been validated. Without wishing to be bound by theory, technologies, methods, and/or devices described herein cause a more predictable tissue response, do not have the same restrictions as thermal methods, and may thus be applied to any part of the body. In certain embodiments, technologies, methods, and/or devices described herein comprise treating a site that is located over or near the thyroid gland, thyroid cartilage, trachea, a major blood vessel, or breast tissue.

In certain embodiments, technologies, methods, and/or devices described herein comprise treating a site that is not located over an area that comprises a mechanical implant, a dermal filler, or a breast implant. In certain embodiments, technologies, methods, and/or devices described herein comprise treating a site that is not located over or near the thyroid gland, thyroid cartilage, trachea, a major blood vessel, or breast tissue.

### Exemplary Conditions to be Treated

Technologies, methods, and/or devices described herein can be used to treat or ameliorate a large number of conditions as described above.

In certain embodiments, technologies, methods, and/or devices described herein can be used for cosmetic purposes, particularly skin rejuvenation. Generally, skin rejuvenation refers to the removal or reduction of blotches, scars, wrinkles, or lines in the skin, particularly the face. One method is the surgical removal of excess skin. This method carries all the risks and side effects of surgery, such as prolonged healing, risk of infection, and scarring. An alternative is thermal ultrasound therapy. The aim of thermal ultrasound is to bypass the surface of the skin to deliver an effective amount of ultrasound energy at certain target depths. This thermal energy aims to trigger a natural response under the skin, jumpstarting the regenerative process that produces collagen, thus rejuvenating the skin. Side effects of this therapy may include redness, swelling, discomfort, bruising, nerve damage, and scarring. Thermal ablation, e.g., using fractional CO₂ lasers, is widely used for skin rejuvenation. Laser ablation involves the layer-by-layer removal of skin, with the aim that the skin cells formed during healing give the skin a tighter and younger appearance. It has been shown, however, that complications with fractional laser skin resurfacing, e.g., post-inflammatory hyperpigmentation prolonged erythema, skin swelling, and infection, are common and can cover the full spectrum of severity and duration (see Zhu et al., BioMed Research International, vol. 2016). In general, it has been shown that a greater likelihood of developing post-treatment complications can be observed in sensitive cutaneous areas and in patients with intrinsically darker skin phototypes or predisposing medical risk factors (see Metelitsa et al., Dermatol Surg. 2010 Mar; 36(3):299-306). Technologies, methods, and/or devices described herein can be used to rejuvenate skin avoiding the risks and side effects associated with thermal methods by removing microcores from skin, causing the skin to tighten as the microscopic holes are closed.

Technologies, methods, and/or devices described herein can be used for treatment of scars. Scars are characterized by fibroblast proliferation and overexpression of collagen that crosslinks and aligns in one specific direction. Scar tissue is often inferior to healthy tissue, e.g., scars in the skin are less resistant to ultraviolet radiation, lack sweat glands and hair follicles, and are of inferior appearance. Scars can be caused by a variety of conditions, e.g., trauma, abrasion, acne etc., on a variety of tissues. Current treatment of scars include chemical peels, filler injections (e.g. collagen), dermabrasion, laser treatment, radiotherapy, dressing, and steroids, all of which can have significant limitations and/or side effects. Technologies, methods, and/or devices herein can be used to debulk scars by removing microcores from the scar, thus breaking up the hardened tissue and allowing healthy tissue to grow into the microcavities. For example, technologies, methods, and/or devices described herein can be applied to scars on skin or other tissue, such as muscles, e.g., scars on the heart muscle after myocardial infarction.

Technologies, methods, and/or devices described here in can be used for removal of tattoos. During tattooing, skin is penetrated by a needle carrying ink, and ink particles are inserted into the dermis. After the healing process, the ink pigment remains trapped within fibroblasts, ultimately concentrating in a layer just below the dermis/epidermis boundary, where it remains stable. Tattoo removal techniques involve using lasers to break up the pigments, upon which they are cleared by the body's immune system. A major obstacle is the fact that lasers are color sensitive. Thus, for multi-colored tattoos, repeated procedures are usually necessary. The procedures are often painful and carry risks of side effects, such as scarring, keloid formation, and hypopigmentation. Technologies, methods, and/or devices described herein can be used to remove tattoos by physically removing tissue containing pigment.

### Subjects

Technologies, methods, and/or devices described herein can be used on any subject. In certain embodiments, a subject is an animal, wherein the animal can be a mammal, wherein the mammal can be a human.

In some embodiments, subjects of any age can be treated with technologies, methods, and/or devices described herein, e.g., adult subjects underdoing aesthetic wrinkle treatment or children undergoing scar remodeling and treatment. In certain embodiments, a subject is under the age of 10 years. In certain embodiments, a subject is over the age of 100 years. In certain embodiments, a subject is between 10 and 100 years, between 15 and 90 years, between 20 and 85 years, between 25 and 80 years, between 30 and 75 years, or between 40 and 70 years of age.

In some embodiments, subjects of any skin type can be treated with technologies, methods, and/or devices described herein. In certain embodiments, a subject is light-skinned. In certain embodiments, a subject is dark-skinned. In certain embodiments, a subject has Fitzpatrick Skin Type 1, 2, or 3. In certain embodiments, a subject has Fitzpatrick Skin Type 4, 5, or 6. Technologies, methods, and/or devices described herein are particularly well suited for patients with Fitzpatrick Skin Type 4, 5, or 6, as these subjects are prone to experience photobleaching when undergoing photodynamic or thermal therapy.

In some embodiments, subjects with any condition of any facial skin fold or wrinkle category can be treated with technologies, methods, and/or devices described herein. In certain embodiments, a subject has re-auricular wrinkle severity graded as ≥1, ≥2, ≥3, ≥4, or ≥5 (See FIG. 1). In certain embodiments, a subject has re-auricular wrinkle severity graded as ≥2 and ≤4, or ≥1 and ≤5. In certain embodiments, a subject has nasolabial fold severity at rest ≥1, ≥2, ≥3, ≥4, or ≥5 (See FIG. 2). In certain embodiments, a subject has nasolabial fold severity at rest ≥2 and ≤4, or ≥1 and ≤5. In certain embodiments, a subject has marionette line prominence at rest 1, ≥2, ≥3, ≥4, or ≥5 (See FIG. 3). In certain embodiments, a subject has marionette line prominence at rest ≥2 and ≤4, or ≥1 and ≤5. In certain embodiments, a subject has oral commissure drooping at rest ≥1, ≥2, ≥3, ≥4, or ≥5 (See FIG. 4). In certain embodiments, a subject has oral commissure drooping at rest ≥2 and ≤4, or ≥1 and ≤5. In certain embodiments, a subject has jawline sagging at rest ≥1, ≥2, ≥3, ≥4, or ≥5 (See FIG. 5). In certain embodiments, a subject has jawline sagging at rest ≥2 and ≤4, or ≥1 and ≤5.

In some embodiments, a subject to be treated with the technologies, methods, and/or devices described herein does not have any of: lesions suspicious for any malignancy or the presence of actinic keratosis, melasma, vitiligo, cutaneous papules/nodules or active inflammatory lesions in the areas to be treated; history of keloid formation or hypertrophic scarring.

In some embodiments, a subject to be treated with technologies, methods, and/or devices described herein does not have any of: history of trauma or surgery to the treatment areas; scar present in the areas to be treated; silicone or synthetic material injections in the areas to be treated; injection of FDA-approved dermal fillers in the past two years; injection of fat in the past year; history of treatment with dermabrasion, laser, or radiofrequency; history of treatment with botulinum toxin injections in the areas to be treated within the prior 6 months; active, chronic, or recurrent infection; history of compromised immune system or currently being treated with immunosuppressive agents; history of sensitivity to analgesic agents, Aquaphor®, topical or local anesthetics (e.g., lidocaine, benzocaine, procaine) or chlorhexidine, povidone-iodine or epinephrine; excessive sun exposure and use of tanning beds or tanning creams within 30 days prior to treatment; treatment with aspirin or other blood thinning agents within 14 days prior to treatment; history or presence of any clinically significant bleeding disorder; and history of drug and/or alcohol abuse; and is not an active smoker (0.5 pack/day) or has quit within 3 months prior to treatment.

### Timing

Technologies, methods, and/or devices described herein allow for rapid microcoring of any site. Technologies, methods, and/or devices described herein permit a microcoring procedure to be completed within a certain time period that commences at the moment a first microneedle or microcoring device touches a tissue (e.g., the skin) (“first contact”) and ends when a last microneedle or microcoring device is removed from the tissue (“last contact”). In certain embodiments, more than one microneedle or microcoring device touches a tissue simultaneously during first contact. In certain embodiments, more than one microneedle or microcoring device is removed simultaneously from a tissue during last contact. In certain embodiments, a time period is less than about 30 minutes. In certain embodiments, a time period is less than about 1 minute. In certain embodiments, a time period is between 1 second and 1 minute. In certain embodiments, a time period is between 1 minute and 2 hours, between 2 minutes and 1.5 hours, between 3 minutes and 1.2 hours, between 4 minutes and 1 hour, between 5 minutes and 50 minutes, between 6 minutes and 45 minutes, between 7 minutes and 40 minutes, between 8 minutes and 35 minutes, between 9 minutes and 30 minutes, or between 10 minutes and 25 minutes.

In certain embodiments, microcores are excised at a certain rate, e.g., in embodiments comprising a reciprocating microneedle or microcoring implement arrangement (e.g., microneedle arrangement). In certain embodiments, the excising of tissue is performed at a rate of between about 100 to 30,000 cores/minute, between about 120 and about 25,000 cores/minute, about 140 and about 20,000 cores/minute, about 160 and about 15,000 cores/minute, about 180 and about 10,000 cores/minute, about 200 and about 5,000 cores/minute, about 220 and about 4,000 cores/minute, about 220 and about 3,000 cores/minute about, 240 and about 2,000 cores/minute, about 260 and about 1,000 cores/minute, about 280 and about 900 cores/minute, about 300 and about 800 cores/minute, about 320 and about 700 cores/minute, about 340 and about 600 cores/minute, about 360 and about 500 cores/minute, or about 380 and about 400 cores/minute.

### Healing and Outcome

In certain embodiments, technologies, methods, and/or devices provided herein offer particularly useful and/or effective microcoring strategies. In certain embodiments, technologies, methods, and/or devices provided herein are characterized by one or more desirable healing attributes. The microcoring technologies, methods, and/or devices described herein can have positive effects on healing, e.g., through selection of an appropriate core depth, size, and/or pattern. Healing and positive outcomes may be accelerated using appropriate pre-treatment and/or post-treatment technologies, methods, and/or devices, as described above. In certain embodiments, application of technologies, methods, and/or devices provided herein can comprise a subject experiencing improvements skin appearance and/or to rejuvenation of skin immediately after completion of treatment. In certain embodiments, application of technologies, methods, and/or devices provided herein can comprise a subject, on Day 3 post treatment, experiencing ecchymosis, tenderness, pruritis, erythema/inflammation, crusting, hyper pigmentation, hypo pigmentation, swelling/fluid accumulation, and/or bleeding at an average severity level of below 1.5 (on a 0-4 severity scale). In certain embodiments, application of technologies, methods, and/or devices provided herein can comprise a subject, on Day 5 post treatment, experiencing ecchymosis, tenderness, pruritis, erythema/inflammation, crusting, hyper pigmentation, hypo pigmentation, swelling/fluid accumulation, and/or bleeding, at an average severity level of below 1.5 (on 0-4 severity scale). Severity of a skin condition can be determined, e.g., via visual inspection and allocation of a severity score, e.g., from 0 (no skin changes) to 4 (severe skin change). In addition, an area fraction of a site affected by a skin condition can be determined by visual inspection and/or basic medical imaging techniques. In certain embodiments, application of technologies, methods, and/or devices provided herein can comprise a subject exhibiting no appearance of scarring 3 days, 4 days, 5 days, 6 days, 7 days, 2 weeks, 3 weeks, 4 weeks, 5 weeks, 6 weeks, 2 months, 3 months or 6 months after treatment.

In certain embodiments, application of technologies, methods, and/or devices provided herein can comprise a subject, on Day 7 post treatment, experiencing a global aesthetic improvement scale (GAIS) score of at least 3 (Improved). The GAIS score can be determined by observation and scoring, e.g., on a 5-1 scale wherein, 5=worse; 4=no change; 3=Improved; 2=Much Improved; 1=Exceptionally improved (see e.g., Han et al, Arch Aesthetic Plast Surg 2014; 20(3):160-164).

In certain embodiments, application of technologies, methods, and/or devices provided herein can comprise a subject, on Day 7 post treatment, experiencing re-auricular wrinkle severity improved by at least 1 level; Nasolabial fold severity at rest improved by at least 1 level; Marionette line prominence at rest improved by at least 1 level; Oral commissure drooping at rest improved by at least 1 level; and/or Jawline sagging at rest improved by at least 1 level. In certain embodiments, application of technologies, methods, and/or devices provided herein can comprise a subject, on Day 7 post treatment, experiencing re-auricular wrinkle severity improved by at least 2 levels; Nasolabial fold severity at rest improved by at least 2 levels; Marionette line prominence at rest improved by at least 2 levels; Oral commissure drooping at rest improved by at least 2 levels; and/or Jawline sagging at rest improved by at least 2 levels. In certain embodiments, application of technologies, methods, and/or devices provided herein can comprise a subject, on Day 7 post treatment, experiencing re-auricular wrinkle severity improved by at least 3 levels; Nasolabial fold severity at rest improved by at least 3 levels; Marionette line prominence at rest improved by at least 3 levels; Oral commissure drooping at rest improved by at least 3 levels; and/or Jawline sagging at rest improved by at least 3 levels. The determination of improvement levels is described above.

In certain embodiments, application of technologies, methods, and/or devices provided herein can comprise a subject experiencing a significantly reduced amount of swelling, bruising, and/or pain compared with the amount of swelling, bruising, and/or pain associated with a standard surgical procedure (e.g., face lift) treating the same condition. In certain embodiments, application of technologies, methods, and/or devices provided herein can comprise a subject being able to return to work post-treatment within a significantly shorter time period compared with the time period associated with a standard surgical procedure (e.g., face lift) treating the same condition.

### Drug Delivery

In general, in some embodiments, one or more therapeutic agents may be delivered and/or administered as part of or in conjunction with one or more technologies, methods and/or devices as described herein. In general, a therapeutic agent may be delivered by any appropriate or feasible route of administration (e.g., topical, enteral, parenteral, etc). In some embodiments, a therapeutic agent may be administered before, during, and/or after part or all of a procedure as described herein.

In some particular embodiments, technologies, methods, and/or devices described herein can be used to deliver one or more therapeutic agents to a treatment site. Among other things, the present disclosure encompasses the recognition that certain technologies as described herein have attributes that render them particularly advantageous for drug delivery. For example, the present disclosure encompasses the recognition that certain heat-based strategies to treating tissue can induce coagulation, cause scarring, and/or have other effects that can inhibit or interfere with drug delivery. By contrast, microcoring strategies as described herein do not cause such heat effects. Furthermore, provided microcoring strategies can provide uniformity in delivery setting (e.g., via substantial uniformity in dimensions—e.g., diameter and/or depth-of core site). In addition, microcoring strategies as described can create a channel through the complete thickness of the dermis, whereas laser technologies typically only ablate part of the dermis.

In some embodiments, hollow needles described herein may be configured and/or procedures may be performed so that one or more therapeutic agents is delivered

In some embodiments, hollow needles may be capable of creating direct channels or holes to the local blood supply and local perfusion by removing cored tissue portions. Direct channels or holes may be used to deliver useful therapeutic agents. Depending on the size (e.g., diameter and/or active length) of hollow needles, holes having different diameters and/or penetration depths may be created. For example, hollow needles having a large diameter (e.g., 18 gauge) and/or a long active length may be used to create large and deep holes that may be used as delivery channels to deliver a large volume dose of one or more therapeutic agents.

In some embodiments, a therapeutic agent may be delivered by injection through a hollow needle. In some such embodiments, an injected composition comprising a therapeutic agent may be or comprise a liquid, a gel, a semi-solid, or a solid. In some embodiments, an injected composition may be or comprise an extended release formulation (e.g., a depot formulation); in some embodiments, an injected composition may be or comprise an immediate release formulation. Alternatively or additionally, in some embodiments, an injected composition may be or comprise a delayed release composition.

In some embodiments, a therapeutic agent may be delivered by needle impregnation and/or coating, so that the agent is released from the needle or a surface thereof.

The present disclosure further encompasses the recognition that, in some embodiments, release or escape of a therapeutic agent administered via a microhole as described herein may be reduced, for example, by restricting one or more feature or avenue of its escape or release from the hole. In some embodiments, for example, holes may be plugged. In some embodiments, holes may be covered with a dressing (e.g., a compressive or occlusive dressing) and/or a closure (e.g., bandages, hemostats, sutures, or adhesives), for example to prevent or limit a delivered therapeutic agent from leaking out of the skin and/or to maintain moisture of a treated skin area.

Delivery of a therapeutic agent through the holes created by hollow needles may provide precise control of dosing of therapeutic agents. In certain embodiments, such dosing may be more accurate than dosing using techniques employing solid microneedles due to the challenges in coating solid needles with a therapeutic agent and controllably releasing such agent. Without wishing to be bound by theory, because technologies, methods, and/or devices described herein do not cauterize the wounds created therewith, perfusion of tissue is maintained and a therapeutic agent or agents may enter the blood stream unimpeded. In certain embodiments, a therapeutic agent may be delivered in form of a liquid, a gel, a matrix, a powder, a microparticle, a nanoparticle, or an aerosol.

Examples of useful therapeutic agents include one or more growth factors (e.g., vascular endothelial growth factor (VEGF), platelet-derived growth factor (PDGF), transforming growth factor beta (TGF-β), fibroblast growth factor (FGF), epidermal growth factor (EGF), and keratinocyte growth factor); one or more stem cells (e.g., adipose tissue-derived stem cells and/or bone marrow-derived mesenchymal stem cells); one or more skin whitening agents (e.g., hydroquinone); one or more vitamin A derivatives (e.g., tretinoin), one or more analgesics (e.g., paracetamol/acetaminophen, aspirin, a non steroidal antiinflammatory drug, as described herein, a cyclooxygenase-2-specific inhibitor, as described herein, dextropropoxyphene, co-codamol, an opioid (e.g., morphine, codeine, oxycodone, hydrocodone, dihydromorphine, pethidine, buprenorphine, tramadol, or methadone), fentanyl, procaine, lidocaine, tetracaine, dibucaine, benzocaine, p-butylaminobenzoic acid 2-(diethylamino) ethyl ester HCl, mepivacaine, piperocaine, dyclonine, or venlafaxine); one or more antibiotics (e.g., cephalosporin, bactitracin, polymyxin B sulfate, neomycin, bismuth tribromophenate, or polysporin); one or more antifungals (e.g., nystatin); one or more antiinflammatory agents (e.g., a non-steroidal anti-inflammatory drug (NSAID, e.g., ibuprofen, ketoprofen, flurbiprofen, piroxicam, indomethacin, diclofenac, sulindac, naproxen, aspirin, ketorolac, or tacrolimus), a cyclooxygenase-2-specific inhibitor (COX-2 inhibitor, e.g., rofecoxib (Vioxx®), etoricoxib, and celecoxib (Celebrex®)), a glucocorticoid agent, a specific cytokine 25 directed at T lymphocyte function), a steroid (e.g., a corticosteroid, such as a glucocorticoid (e.g., aldosterone, beclometasone, betamethasone, cortisone, deoxycorticosterone acetate, dexamethasone, fludrocortisone acetate, hydrocortisone, methylprednisolone, prednisone, prednisolone, or triamcinolone) or a mineralocorticoid agent (e.g., aldosterone, corticosterone, or deoxycorticosterone)), or an immune selective antiinflammatory derivative (e.g., phenylalanine-glutamine-glycine (FEG) and its D-isomeric form (feG))); one or more antimicrobials (e.g., chlorhexidine gluconate, iodine (e.g., tincture of iodine, povidone-iodine, or Lugol's iodine), or silver, such as silver nitrate (e.g., as a 0.5% solution), silver sulfadiazine (e.g., as a cream), or Ag+ in one or more useful carriers (e.g., an alginate, such as Acticoat® including nanocrystalline silver coating in high density polyethylene, available from Smith & Nephew, London, U.K., or Silvercel® including a mixture of alginate, carboxymethylcellulose, and silver coated nylon fibers, available from Systagenix, Gatwick, U.K.; a foam (e.g., Contreet® Foam including a soft hydrophilic polyurethane foam and silver, available from Coloplast A/S, Humlebæk, Denmark); a hydrocolloid (e.g., Aquacel® Ag including ionic silver and a hydrocolloid, available from Conva Tec Inc., Skillman, NJ); or a hydrogel (e.g., Silvasorb® including ionic silver, available from Medline Industries Inc., Mansfield, MA)); one or more antiseptics (e.g., an alcohol, such as ethanol (e.g., 60-90%), 1-propanol (e.g., 60-70%), as well as mixtures of 2-propanol/isopropanol; boric acid; calcium hypochlorite; hydrogen peroxide; manuka honey and/or methylglyoxal; a phenol (carbolic acid) compound, e.g., sodium 3,5-dibromo-4-hydroxybenzene sulfonate, trichlorophenylmethyl iodosalicyl, or triclosan; 5 a polyhexanide compound, e.g., polyhexamethylene biguanide (PHMB); a quaternary ammonium compound, such as benzalkonium chloride (BAC), benzethonium chloride (BZT), cetyl trimethylammonium bromide (CTMB), cetylpyridinium chloride (CPC), chlorhexidine (e.g., chlorhexidine gluconate), or octenidine (e.g., octenidine dihydrochloride); sodium bicarbonate; sodium chloride; sodium hypochlorite (e.g., optionally in combination with boric acid in Dakin's solution); or a triarylmethane dye (e.g., Brilliant Green)); one or more antiproliferative agents (e.g., sirolimus, tacrolimus, zotarolimus, biolimus, or paclitaxel); one or more emollients; one or more hemostatic agents (e.g., collagen, such as microfibrillar collagen, chitosan, calcium-loaded zeolite, cellulose, anhydrous aluminum sulfate, silver nitrate, potassium alum, titanium oxide, fibrinogen, epinephrine, calcium alginate, poly-N-acetyl glucosamine, thrombin, coagulation factor(s) (e.g., II, V, VII, VIII, IX, X, XI, XIII, or Von Willebrand factor, as well as activated forms thereof), a procoagulant (e.g., propyl gallate), an anti-fibrinolytic agent (e.g., epsilon aminocaproic acid or tranexamic acid), and the like); one or more procoagulative agents (e.g., any hemostatic agent described herein, desmopressin, coagulation factor(s) (e.g., II, V, VII, VIII, IX, X, XI, XIII, or Von Willebrand factor, as well as activated forms thereof), procoagulants (e.g., propyl gallate), antifibrinolytics (e.g., epsilon aminocaproic acid), and the like); one or more anticoagulative agents (e.g., heparin or derivatives thereof, such as low molecular weight heparin, fondaparinux, or idraparinux; an anti-platelet agent, such as aspirin, dipyridamole, ticlopidine, clopidogrel, or prasugrel; a factor Xa inhibitor, such as a direct factor Xa inhibitor, e.g., apixaban or rivaroxaban; a thrombin inhibitor, such as a direct thrombin inhibitor, e.g., argatroban, bivalirudin, dabigatran, hirudin, lepirudin, or ximelagatran; or a coumarin derivative or vitamin K antagonist, such as warfarin (coumadin), acenocoumarol, atromentin, phenindione, or phenprocoumon); one or more immune modulators, including corticosteroids and non-steroidal immune modulators (e.g., NSAIDS, such as any described herein); one or more proteins; and/or one or more vitamins (e.g., vitamin A, C, and/or E). One or more of botulinum toxin, fat (e.g. autologous), hyaluronic acid, a collagen-based filler, or other filler may also be administered to the skin. Platelet rich plasma may also be administered to the skin. One or more therapeutic agents described herein may be formulated as a depot preparation. In general, depot preparations are typically longer acting than non-depot preparations. In some embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a therapeutic agent may include an anticoagulative and/or procoagulative agent. For instance, by controlling the extent of bleeding and/or clotting in treated skin regions, a skin tightening effect may be more effectively controlled. Thus, in certain embodiments, technologies, methods, and/or devices herein include or can be used to administer one or more anticoagulative agents, one or more procoagulative agents, one or more hemostatic agents, one or more fillers, or a combination thereof. In particular embodiments, a therapeutic agent controls the extent of bleeding, bruising, and/or clotting in the treated skin region, including the use one or more anticoagulative agents (e.g., to inhibit clot formation prior to skin healing or slit/hole closure) and/or one or more hemostatic or procoagulative agents. In certain embodiments, a therapeutic agent includes a hemostatic agent (e.g., epinephrine) to control bleeding and/or bruising.

In certain embodiments, a therapeutic agent may be delivered transdermally, intradermally, locally, subcutaneously, or in a combination thereof. In some embodiments, a therapeutic agent can be delivered in conjunction with a micocoring procedure for scar debulking. Without wishing to be bound by theory, this method would comprise removing an amount of scar tissue and the contemporaneous or subsequent administration of drug inhibiting or preventing new scar formation. Alternatively or additionally, another agent such as botox, antibiotics, anti-inflammatoirealing promoters (inhibit scar formation) may be administered. Alternatively or additionally, tissue bulking fat/plumping materials (e.g., particularly when scar removal) may be administered. These steps may occur contemporaneously or in any sequence.

### Combination Therapies

In certain embodiments, technologies, methods, and/or devices provided herein can be used in combination with other technologies, methods and/or devices, e.g., non-thermal or thermal skin rejuvenation technologies. Without wishing to be bound by theory, application of the technologies, methods, and/or devices provided herein in combination with other, potentially more harmful and/or invasive technologies may greatly reduce the undesired effects and/or increase the cosmetic effect of such harmful and/or invasive technologies. In certain embodiments, technologies, methods, and/or devices provided herein can be used in combination with non-invasive fat removal technologies, e.g., Coolsculpting® by Zeltiq® or Sculpsure® by Cynosure®. In certain embodiments, technologies, methods, and/or devices provided herein can be used in combination with Invasive fat removal technologies, e.g., standard liposuction or energy-assisted liposuction techniques, e.g., Smartlipo by Cynosure (laser assisted lipolysis), or VASER® by Solta Medical®. In certain embodiments, technologies, methods, and/or devices provided herein can be used in combination with tightening technologies that deposit energy underneath the skin, e.g., Ultherapy® by Ulthera® (ultrasound energy), Thermage® by Solta Medical® (RF energy). In certain embodiments, technologies, methods, and/or devices provided herein can be used in combination with Invasive cellulite treatments, e.g., Cellfina® by Merz®, or Cellulaze® by Cynosure®.

### EXEMPLIFICATION

### Example 1: Rapid Microcoring of Facial Sites

The present Example demonstrates certain embodiments of rapid microcoring in accordance with the present disclosure. In particular, the present Example describes certain rapid microcoring technologies that can achieve scarless removal of excess skin, e.g., from certain facial sites.

In this Example, provided technologies are applied to subjects with pre-auricular wrinkles meeting the Inclusion Criteria below (Part A) and/or mid-and lower-face skin laxity manifested by one or more of the following: deepening of the nasolabial folds at rest; prominence of marionette lines at rest; downturn of the oral commissures at rest, sagging of the skin at the jawline at rest meeting the Inclusion Criteria below (Part B).

Part A: Subjects meeting the Inclusion Criteria for bilateral pre-auricular wrinkles on the Lemperle Assessment Scale (see FIG. 1) and mid-and lower-face laxity (as defined above) will be randomized to removal of pre-auricular wrinkles with 22 G and 24 G micro-coring needles at densities (percent of skin removed per 1 cm²) of 5%, 7.5% or 10% on the left and right pre-auricular areas.

At the Day 30 visit, the pre-auricular treatment areas will be assessed for untoward healing outcomes (e.g., scarring, pigmentary changes) and local adverse events. If, in the Investigator's opinion, the subject's left and right treated areas are healed without evidence of untoward healing outcomes and no local adverse events have been noted, which in the Investigator's opinion would make further treatment inadvisable, the subject will be eligible to enter Part B of this study.

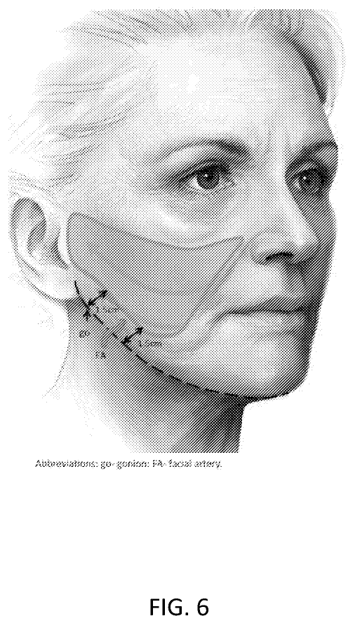

Part B: Subjects will undergo bilateral micro-coring needle scarless removal of excess skin in an area outside of the pre-auricular areas treated in Part A as described by imaginary lines as follows:
from the junction of the superior anterior helix of the ear and the pre-auricular area extending
medially to the superior border of the nasolabial fold at the junction of the nasal ala and the cheek, then
Inferiorly along the nasolabial fold and slightly lateral to the oral commissure to 1.5 cm above the inferior border of the mandible and
posteriorly to the junction of the ear lobule and the cheek (See FIG. 6).

In both Part A and Part B there will be 3 subject cohorts based on treatment density (percent of tissue removed per 1 cm²): 5%, 7.5%, and 10%. In Part A and Part B the cohorts will be treated in escalating densities with subjects enrolled in the next higher density cohort following review of the safety and wound healing profile collected at the Day 7 visit of subjects in the completed cohort. If the safety and wound healing profiles of the cohort are deemed to be satisfactory by the Investigator, the next higher density cohort will be enrolled and treated.

Each subject will have 2 treatment areas (left and right) randomized to 1 needle gauge and density. The same needle gauge and density will be used for the subject in Part A and Part B.
24 G needle, 5% density (5 subjects)
22 G needle, 5% density (5 subjects)
24 G needle, 7.5% density (5 subjects)
22 G needle, 7.5% density (5 subjects)
24 G needle, 10% density (5 subjects)
22 G needle, 10% density (5 subjects)

Parts A and B: Micro-core biopsies of treated pre-auricular and mid-lower-face areas and adjacent untreated control areas will be performed on Day 180 in subjects that consent to the procedure.

Histology of treated and untreated control areas will be compared using standard and special histological techniques (see “Exploratory Endpoints” below).

Study endpoints include certain safety endpoints, effectiveness endpoints, and exploratory endpoints.

Safety Endpoints include the incidence and severity of systemic and local adverse events will be recorded at all visits.

Effectiveness Endpoints include the overall aesthetic improvement. These are assessed via Subject reported Global Aesthetic Improvement Scale (Parts A and B), Investigator reported Global Aesthetic Improvement Scale (Parts A and B), and Subject Satisfaction Scale (Parts A and B).

Effectiveness Endpoints include for Part A only: ≥1 point reduction of the Lemperle Scale as judged by a Live independent, blinded reviewer at each study site at Baseline and Day 30, 90 and 180 visits. Photographs will be taken with a Canfield Visia to document the appearance of treated areas at these visits; and ≥1 point reduction of the Lemperle Assessment Scale as judged by a 3 member, blinded independent review committee comparing photographs at Baseline to Day 30, 90, and 180 visits at study completion (all subjects, last visit).

Effectiveness Endpoints include for Part B: Live evaluation of change in Lower Face Scales by independent, blinded reviewer at Baseline, and Day 30, 90, 180 visits; Evaluation of photographs of Lower Face Scales by 3 member independent, blinded review committee at Baseline and Day 30, 90 and 180 visits; and Evaluation of subjects, whether by Live independent review or by 3 member committee will be conducted at Baseline and Day 30, 90 and 180 visits using the following scales:
≥1 point reduction of the nasolabial fold scale score at rest as described in FIG. 2;
≥1 point reduction of the marionette line scale score at rest as described in FIG. 3;
≥1 point reduction of the oral commissure scale score at rest as described in FIG. 4;
≥1 point reduction of the jawline scale score at rest described in FIG. 5.

Photographs will be taken with a Canfield Visia to document the appearance of treated areas at these visits.

For the evaluation of exploratory endpoints histology of micro-core skin biopsies taken from the treated areas and adjacent untreated skin with a 21 G needle will be examined for epidermal and dermal thickness, collagen types I and III, elastin, myofibroblasts, vinculin, Perls' Prussian Blue for iron, and Fontana Masson for melanin. For Part A only: Measurement of treatment area topography may be assessed using Canfield Primos Lite 45 X 30 performed on selected subjects at selected study sites. For Part B only: Mid-and lower-face skin areal reduction calculated from Canfield Vectra H-1 measurements performed on selected subjects at selected study sites

Inclusion criteria for this study are as follows:
Males and females 40-70 years of age.
Fitzpatrick Skin Type 1, 2, or 3 as judged by the Investigator.
Pre-auricular wrinkle severity graded as ≥2 as judged by the Investigator using the Lemperle Assessment Scale (see FIG. 1) and one or more of the following:
   Nasolabial fold severity at rest ≥2 and ≤4 as assessed by the Investigator using the scale in FIG. 2;
   Marionette line prominence at rest ≥2 and ≤4 as assessed by the Investigator using the scale in FIG. 3;
   Oral commissure drooping at rest ≥2 and ≤4 as assessed by the Investigator using the scale represented in FIG. 4;
   Jawline sagging at rest ≥2 and ≤4 as assessed by the Investigator using the scale represented in FIG. 5.
Able to provide written informed consent, understand and willing to comply with all study related procedures and follow-up visits
Signed informed consent obtained before any study-specific procedure is performed.

Exclusion criteria for this study are as follows:
Lesions suspicious for any malignancy or the presence of actinic keratosis, melasma, vitiligo, cutaneous papules/nodules or active inflammatory lesions in the areas to be treated
History of keloid formation or hypertrophic scarring
History of trauma or surgery to the treatment areas
Scar present in the areas to be treated
Silicone or synthetic material injections in the areas to be treated
Injection of FDA-approved dermal fillers in the past two years
Injection of fat in the past year
History of treatment with dermabrasion, laser, or radiofrequency
History of treatment with botulinum toxin injections in the areas to be treated within the prior 6 months
Active smokers (0.5 pack/day) or having quit within 3 months prior to treatment
Active, chronic, or recurrent infection
History of compromised immune system or currently being treated with immunosuppressive agents
History of sensitivity to analgesic agents, Aquaphor®, topical or local anesthetics (e.g., lidocaine, benzocaine, procaine) or chlorhexidine, povidone-iodine or epinephrine
Excessive sun exposure and use of tanning beds or tanning creams within 30 days prior to treatment
Treatment with aspirin or other blood thinning agents within 14 days prior to treatment
History or presence of any clinically significant bleeding disorder
Co-morbid condition that in the Investigator's opinion could limit ability to participate in the study or to comply with follow-up requirements
History of drug and/or alcohol abuse
Any issue that, at the discretion of the Investigator, would interfere with assessment of safety or efficacy or compromise the subject's ability to undergo study procedures or give informed consent
Treatment with an investigational device or agent within 30 days before treatment. or during the study period.

### REFERENCES

Alkilani et al., Transdermal Drug Delivery: Innovative Pharmaceutical Developments Based on Disruption of the Barrier Properties of the stratum corneum, Pharmaceutics, Vol. 7, No. 4, 2015.
Fabi, Noninvasive skin tightening: focus on new ultrasound techniques, Clin Cosmet Investig Dermatol. Vol 8, (2015).
Goldberg et al., Skin Rejuvenation with Non-Invasive Pulsed Electric Fields, Sci Rep, Vol. 5, (2015).
Han et al., Combined, Minimally Invasive, Thread-based Facelift, Arch Aesthetic Plast Surg, Vol. 20, No. 3, 2014
Lee et al., Combined Treatment with Botulinum Toxin and 595-nm Pulsed Dye Laser for raumatic Scarring, Ann Dermatol, Vol. 27, No. 6, 2015.
Paithankar et al., Acne Treatment Based on Selective Photothermolysis of Sebaceous Follicles with Topically Delivered Light-Absorbing Gold Microparticles. Journal of Invest Dermatol, Vol. 135(2015 ).
Wong et al., Hypopigmentation Induced by Frequent Low-Fluence, Large-Spot-Size QS Nd:YAG Laser Treatments, Ann Dermatol, Vol. 27, No. 6, 2015.
Zhu et al., The Efficacy and Safety of Fractional CO₂ Laser Combined with Topical Type A Botulinum Toxin for Facial Rejuvenation: A Randomized Controlled Split-Face Study, Hindawi BioMed Research International, Vol. 2016(2016 ).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A method comprising steps of:
excising a plurality of microcores from a site on a surface of a human subject, wherein each of the microcores is characterized by a diameter of between 0.1 mm and 1.0 mm, and/or a volume of between 0.001 mm³ and 6.3 mm³, wherein the excising is completed within a time period between 1 minute and 2 hours.
